(19) United States
(12) Patent Application Publication
(10) Pub. No.: US 2011/0046385 A1
(43) Pub. Date: Feb. 24, 2011

(54) Polymorphs Of Prostaglandin Agonists And Methods For Making The Same

(75) Inventors: David B. Damon, Mystic, CT (US); Jinyang Hong, Stonington, CT (US); Joseph F. Krzyzaniak, Pawcatuck, CT (US)

Correspondence Address: PFIZER INC.;PATENT DEPARTMENT, Bld 114 M/S 9114, EASTERN POINT ROAD, GROTON, CT, 06340, US

(73) Assignee: PFIZER PRODUCTS INC., Groton, CT (US)

(21) Appl. No.: 12/675,175

(22) PCT Filed: Aug. 25, 2008
(86) PCT No.: PCT/IB2008/002252

§ 371 Date: Sep. 22, 2010

Related U.S. Application Data
(60) Provisional application No. 60/968,771, filed on Aug. 29, 2007.
(60) Provisional application No. 61/083,599, filed on Jul. 25, 2008.

(51) Int. Cl. C07D 213/71

(52) U.S. Cl. 546/293

(57) ABSTRACT

The present invention relates to polymorphic crystalline forms or a non-crystalline form or amorphous of the compound (3-(((4-tert-butyl-benzyl)-(pyridine-3-sulfonyl)-amino)-methyl)-phenoxy)-acetic acid sodium salt or a hydrate thereof together with processes for preparing, methods for using, and pharmaceutical compositions containing the same. The invention also relates to substantially pure polymorphic crystalline forms or a non-crystalline form or amorphous form of (3-(((4-tert-butyl-benzyl)-(pyridine-3-sulfonyl)-amino)-methyl)-phenoxy)-acetic acid sodium salt or a hydrate thereof.

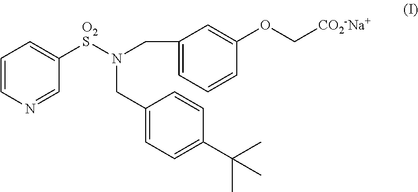

### FIELD OF THE INVENTION

The present invention relates to polymorphs of therapeutically active and selective modulators of prostaglandin, specifically agonists of EP₂, pharmaceutical compositions comprising these compounds, methods for the preparation of these compounds and the use of such compounds for treating conditions that are associated with the modulation of prostaglandin, such as bone disorders, glaucoma and ocular hypertension. More specifically, the present invention relates to polymorphs of (3-(((4-tert-butyl-benzyl)-(pyridine-3-sulfonyl)-amino)-methyl)-phenoxy)-acetic acid sodium salt, pharmaceutical compositions comprising polymorphs of this compound, methods for the preparation of these polymorphs and the use of polymorphs of this compound for treating conditions that are associated with the modulation of prostaglandin.

### BACKGROUND OF THE INVENTION

In the course of drug development, it is generally assumed to be important to discover the most stable crystalline form of the drug. This most stable crystalline form is the form which is likely to have the best chemical stability, and thus the longest shelf-life in a formulation. However, it is also advantageous to have multiple forms of a drug, e.g. salts, hydrates, polymorphs, crystalline and noncrystalline forms. There is no one ideal physical form of a drug because different physical forms provide different advantages. The search for the most stable form and for such other forms is arduous and the outcome is unpredictable.

The successful development of a drug requires that it meet certain requirements to be a therapeutically effective treatment for patients. These requirements fall into two categories: (1) requirements for successful manufacture of dosage forms and (2) requirements for successful drug delivery and disposition after the drug formulation has been administered to the patient.

There are many kinds of drug formulations for administration by various routes, and the optimum drug form for different formulations is likely to be different. As mentioned above, a drug formulation must have sufficient shelf life to allow successful distribution to patients in need of treatment. In addition, an oral drug formulation must provide the drug in a form which will dissolve in the patient's gastrointestinal tract when orally dosed. For oral dosing in an immediate release dosage form, such as an immediate release tablet, capsule, suspension, or sachet, it is generally desirable to have a drug salt or drug form which has high solubility, in order to assure complete dissolution of the dose and optimal bioavailability. For some drugs, particularly low solubility drugs or poorly wetting drugs, it may be advantageous to utilize a noncrystalline drug form, which will generally have a higher initial solubility than a crystalline form when administered into the gastrointestinal tract. A noncrystalline form of a drug is frequently less chemically stable than a crystalline form. Thus, it is advantageous to identify noncrystalline drug forms which are sufficiently chemically stable to provide a practical product which is stable enough to maintain its potency for enough time to permit dosage form manufacture, packaging, storage, and distribution to patients around the world.

On the other hand, there are dosage forms which operate better if the drug form is less soluble. For example, a chewable tablet or a suspension or a sachet dosage form exposes the tongue to the drug directly. For such dosage forms, it is desirable to minimize the solubility of the drug in the mouth, in order to keep a portion of the drug in the solid state, minimizing bad taste. For such dosage forms, it is often desirable to use a low solubility salt or crystalline form.

For controlled release oral or injectable, e.g. subcutaneous or intramuscular, dosage forms, the desired drug solubility is a complex function of delivery route, dose, dosage form design and desired duration of release. For a drug which has high solubility, it may be desirable to utilize a lower solubility crystalline salt or polymorph for a controlled release dosage form, to aid in achievement of slow release through slow dissolution. For a drug which has low solubility, it may be necessary to utilize a higher solubility crystalline salt or polymorph, or a noncrystalline form, in order to achieve a sufficient dissolution rate to support the desired drug release rate from the controlled release dosage form.

In soft gelatin capsule dosage forms (“soft-gels”), the drug is dissolved in a small quantity of a solvent or vehicle such as a triglyceride oil or polyethylene glycol, and encapsulated in a gelatin capsule. An optimal drug form for this dosage form is one which has a high solubility in an appropriate soft-gel vehicle. In general, a drug form which is more soluble in a triglyceride oil will be less soluble in water. Identification of an appropriate drug form for a soft-gel dosage form requires study of various salts, polymorphs, crystalline and noncrystalline forms.

Thus, it can be seen that the desired solubility of a drug form depends on the intended use and not all drug forms are equivalent.

For a drug form to be practically useful for human or animal therapy, it is desirable that the drug form exhibit minimal hygroscopicity. Dosage forms containing highly hygroscopic drugs require protective packaging, and may exhibit altered dissolution if stored in a humid environment. Thus, it is desirable to identify nonhygroscopic crystalline salts and polymorphs of a drug. If a drug is noncrystalline, or if a noncrystalline form is desired to improve solubility and dissolution rate, then it is desirable to identify a noncrystalline salt or form which has a low hygroscopicity relative to other noncrystalline salts or forms.

A drug, crystalline or noncrystalline, may exist in an anhydrous form or as a hydrate or solvate or hydrate/solvate. The hydration state and solvation state of a drug affects its solubility and dissolution behavior.

The melting point of a drug may vary for different salts, polymorphs, crystalline and noncrystalline forms. To permit manufacture of tablets on commercial tablet presses, it is desirable that the drug melting point be greater than around 60° C., preferably greater than 100° C. to prevent drug melting during tablet manufacture. A preferred drug form in this instance is one that has the highest melting point. In addition, it is desirable to have a high melting point to assure chemical stability of a solid drug in a solid dosage form at high environmental storage temperatures which occur in direct sunlight and in geographic areas such as near the equator. If a soft-gel dosage form is desired, it is preferred to have a drug form which has a low melting point, to minimize crystallization of the drug in the dosage form. Thus, it can be seen that the desired melting point of a drug form depends on the intended use and not all drug forms are equivalent.

When the dose of a drug is high, or if a small dosage form is desired, the selection of a salt, hydrate or solvate affects the potency per unit weight. For example, a drug salt with a higher molecular weight counterion will have a lower drug potency per gram than will a drug salt with a lower molecular weight counterion. It is desirable to choose a drug form which has the highest potency per unit weight.

The method of preparation of different crystalline polymorphs and noncrystalline forms varies widely from drug to drug. It is desirable that minimally toxic solvents be used in these methods, particularly for the last synthetic step, and particularly if the drug has a tendency to exist as a solvate with the solvent utilized in the last step of synthesis. Preferred drug forms are those which utilize less toxic solvents in their synthesis.

The ability of a drug to form good tablets at commercial scale depends upon a variety of drug physical properties, such as the Tableting Indices described in Hiestand H, Smith D. Indices of tableting performance. Powder Technology, 1984; 38:145-159. These indices may be used to identify forms of a drug, e.g. of atorvastatin calcium, which have superior tableting performance. One such index is the Brittle Fracture Index (BFI), which reflects brittleness, and ranges from 0 (good—low brittleness) to 1 (poor—high brittleness). Other useful indices or measures of mechanical properties, flow properties and tableting performance include compression stress, absolute density, solid fraction, dynamic indentation hardness, ductility, elastic modulus, reduced elastic modulus, quasistatic indentation hardness, shear modulus, tensile strength, compromised tensile strength, best case bonding index, worst case bonding index, brittle/viscoelasticbonding index, strain index, viscoelastic number, effective angle of internal friction (from a shear cell test), cohesivity (from a powder avalanche test) and flow variability. A number of these measures are obtained on drug compacts, preferably prepared using a triaxial hydraulic press. Many of these measures are further described in Hancock B, Carlson G, Ladipo D, Langdon B, and Mullarney M. Comparison of the Mechanical Properties of the Crystalline and Amorphous Forms of a Drug Substance. International Journal of Pharmaceutics, 2002; 241:73-85.

Drug form properties which affect flow are important not just for tablet dosage form manufacture, but also for manufacture of capsules, suspensions, and sachets.

The particle size distribution of a drug powder can also have large effects on manufacturing processes, particularly through effects on powder flow. Different drug forms have different characteristic particle size distributions.

From the above discussion, it is apparent that there is no one drug form which is ideal for all therapeutic applications. Thus it is important to seek a variety of unique drug forms, e.g. salts, polymorphs, noncrystalline forms, which may be used in various formulations. The selection of a drug form for a specific formulation or therapeutic application requires consideration of a variety of properties, as described above, and the best form for a particular application may be one which has one specific important good property while other properties may be acceptable or marginally acceptable.

U.S. Pat. No. 6,498,172 B1 discloses the compound (3-(((4-tert-butyl-benzyl)-(pyridine-3-sulfonyl)-amino)-methyl)-phenoxy)-acetic acid as being useful in the treatment of, for example, bone disorders. The application refers in general terms to pharmaceutically acceptable salts and the preparation of the sodium salt is disclosed. The '172 patent, however, neither describes any crystallization procedure nor does the patent discuss any polymorph forms of this compound.

Published International patent applications WO 99/19300 and WO 98/28264 disclose prostaglandin agonists and their use to treat and promote the healing of bone fractures and osteotomies by local application (e.g., to the sites of bone fractures or osteotomies).

S. C. Miller and S. C. Marks, Jr., Bone 14, 143-151 (1993), studied the local stimulation of new bone formation on the periosteal surface of the canine mandible by prostaglandin E₁ (PGE₁) and compared delivery by osmotic minipumps and controlled-release pellets implanted subperiosteally next to the lateral mandibular cortex.

S. C. Marks, Jr. and S. C. Miler, J. Oral Pathol. 17:500-505 (1988), reported that local infusion of PGE1 for 3 weeks at doses of 500 to 2000 μg per week produced a dramatic, localized formation of alveolar bone in the mandible of dogs.

In M-S. Shih and R. W. Norrdin, Am. J. Vet. Res. 48: 828-830 (1986), transverse fractures were made surgically in the ribs of adult beagles, and 0.5 ml of 10% ethanol Tris-buffer vehicle or 0.5 ml of PGE₁ (containing 0.2 mg of PGE₁ in 10% ethanol Tris-buffer) was injected directly into the fracture sites twice a day for 10 days. It was concluded that administration of PGE₁ induced bone matrix formation on the periosteal envelope adjacent to the fracture site and its contralateral matching site.

M-S. Shih and R. W. Norrdin, Calcif. Tissue Int. (1986) 39: 191-197, studied the effect of PGE₁ (0.2 mg/kg in 10% ethanol) injected into the defect site in the tibias of beagles twice a day for 10 days after surgery. It was found that the dogs that had received PGE₁ locally had more periosteal and cortical endosteal bone formation, with an increased amount of osteoid present.

R. Yang, T. Liu and S. Lin-Shiau, Calcif. Tissue Int., 52:57-61 (1993), investigated the effect of daily injections of prostaglandin E2 via the intraosseous route into the metaphysis of the left tibia for 14 days. According to this reference, this dosing regimen resulted in a significant increase of trabecular bone in the metaphysis.

K. Notoya et al., The Journal of Pharmacology and Experimental Therapeutics, 290: 1054-1064 (1999), examined the effect of TAK-778, a novel osteoblast differentiation promoting compound, in sustained-release microcapsules applied locally on skeletal regeneration and bone repair in vivo.

### SUMMARY OF THE INVENTION

According to a further embodiment of the present invention, there is provided a crystalline form of (3-(((4-tert-butyl-benzyl)-(pyridine-3-sulfonyl)-amino)-methyl)-phenoxy)-acetic acid sodium salt or a hydrate thereof.

According to still a further embodiment of the present invention, there is provided a pharmaceutical composition comprising a therapeutically effective amount of a crystalline form of (3-(((4-tert-butyl-benzyl)-(pyridine-3-sulfonyl)-amino)-methyl)-phenoxy)-acetic acid sodium salt or a hydrate thereof, and a pharmaceutically acceptable diluent or carrier.

According to another embodiment of the present invention, there is provided a method for treating a mammal having glaucoma, ocular hypertension or a condition which presents with low bone mass comprising administering to the mammal a therapeutically effective amount of a crystalline form of (3-(((4-tert-butyl-benzyl)-(pyridine-3-sulfonyl)-amino)-methyl)-phenoxy)-acetic acid sodium salt or a hydrate thereof.

According to yet a further embodiment of the present invention, there is provided a method for augmenting and maintaining bone mass in a mammal comprising administering to the mammal a therapeutically effective amount of a crystalline form of (3-(((4-tert-butyl-benzyl)-(pyridine-3-sulfonyl)-amino)-methyl)-phenoxy)-acetic acid sodium salt or a hydrate thereof.

According to still another embodiment of the present invention, there is provided a method for preparing crystalline form A of (3-(((4-tert-butyl-benzyl)-(pyridine-3-sulfonyl)-amino)-methyl)-phenoxy)-acetic acid sodium salt hemi-hydrate the method comprises contacting (3-(((4-tert-butyl-benzyl)-(pyridine-3-sulfonyl)-amino)-methyl)-phenoxy)-acetic acid, as the free acid, with sodium hydroxide in an organic solvent to form a reaction mixture; warming the reaction mixture to a first temperature from about 50 C to about 90 C and holding the first temperature for a period of at least one hour; cooling the reaction mixture to a second temperature from about 15 C to about 25 C; and collecting solids from the reaction mixture. The organic solvent used in the method may be isopropyl acetate being present at about 8 to about 12 ml per mmol of (3-(((4-tert-butyl-benzyl)-(pyridine-3-sulfonyl)-amino)-methyl)-phenoxy)-acetic acid. The sodium hydroxide used in the method may be a 50% aqueous solution of sodium hydroxide present at about 1 to about 1.3 mmol per mmol of (3-(((4-tert-butyl-benzyl)-(pyridine-3-sulfonyl)-amino)-methyl)-phenoxy)-acetic acid.

According to a further embodiment of the present invention, there is provided a non-crystalline form or an amorphous form of (3-(((4-tert-butyl-benzyl)-(pyridine-3-sulfonyl)-amino)-methyl)-phenoxy)-acetic acid sodium salt or a hydrate thereof.

According to still a further embodiment of the present invention, there is provided a pharmaceutical composition comprising a therapeutically effective amount of a non-crystalline form or an amorphous form of (3-(((4-tert-butyl-benzyl)-(pyridine-3-sulfonyl)-amino)-methyl)-phenoxy)-acetic acid sodium salt or a hydrate thereof, and a pharmaceutically acceptable diluent or carrier.

According to another embodiment of the present invention, there is provided a method for treating a mammal having glaucoma, ocular hypertension or a condition which presents with low bone mass comprising administering to the mammal a therapeutically effective amount of a non-crystalline form or amorphous form of (3-(((4-tert-butyl-benzyl)-(pyridine-3-sulfonyl)-amino)-methyl)-phenoxy)-acetic acid sodium salt or a hydrate thereof.

According to yet a further embodiment of the present invention, there is provided a method for augmenting and maintaining bone mass in a mammal comprising administering to the mammal a therapeutically effective amount of a non-crystalline form or amorphous form of (3-(((4-tert-butyl-benzyl)-(pyridine-3-sulfonyl)-amino)-methyl)-phenoxy)-acetic acid sodium salt or a hydrate thereof.

The compound “(3-(((4-tert-butyl-benzyl)-(pyridine-3-sulfonyl)-amino)-methyl)-phenoxy)-acetic acid” refers to the free acid of the compound.

The term “mammal” means animals including, for example, dogs, cats, cows, sheep, horses, and humans. Preferred mammals include humans.

The phrase “substantially pure” refers the relative purity of the desired polymorph and/or salt with respect to the overall amount of compound (that is, the amount of the desired polymorph and/or salt plus any other polymorphs and amorphous forms, salts and free acids). A “substantially pure” polymorphic form should contain at least about 90% of the desired polymorph relative to the overall amount of compound. Preferably, a “substantially pure” polymorphic form should contain at least about 95% of the desired polymorph. In some embodiments of the present invention, a “substantially pure” polymorphic form may contain at least about 99% of the desired polymorph.

The phrase “condition(s) which presents with low bone mass” refers to a condition where the level of bone mass is below the age specific normal as defined in standards by the World Health Organization “Assessment of Fracture Risk and its Application to Screening for Postmenopausal Osteoporosis (1994), Report of a World Health Organization Study Group, World Health Organization Technical Series 843”. Included in “condition(s) which presents with low bone mass” are primary and secondary osteoporosis. Secondary osteoporosis includes glucocorticoid-induced osteoporosis, hyperthyroidism-induced osteoporosis, immobilization-induced osteoporosis, heparin-induced osteoporosis and immunosuppressive-induced osteoporosis. Also included is periodontal disease, alveolar bone loss, post-osteotomy and childhood idiopathic bone loss. The phrase “condition(s) which presents with low bone mass” also includes long term complications of osteoporosis such as curvature of the spine, loss of height and prosthetic surgery.

The phrase “condition(s) which presents with low bone mass” also refers to a mammal, e.g., a mammal, known to have a significantly higher than average chance of developing such diseases as are described above including osteoporosis (e.g., post-menopausal women, and men over the age of 60).

Other bone mass augmenting or enhancing uses include bone restoration, increasing the bone fracture healing rate, replacing bone graft surgery entirely, enhancing the rate of successful bone grafts, bone healing following facial reconstruction, maxillary reconstruction, mandibular reconstruction, craniofacial reconstruction, prosthetic ingrowth, vertebral synostosis, long bone extension and spinal fusion.

The pharmaceutical compositions of the present invention may also be used in conjunction with orthopedic devices known to those skilled in the art such as spinal fusion cages, spinal fusion hardware, internal and external bone fixation devices, screws and pins.

Those skilled in the art will recognize that the term bone mass actually refers to bone mass per unit area which is sometimes (although not strictly correctly) referred to as bone mineral density (BMD).

The term “treating”, “treat” or “treatment” as used herein includes preventative (e.g., prophylactic), palliative and curative treatment.

The term “effective amount” means an amount of a compound or combination of compounds that ameliorates, attenuates or eliminates a particular disease or condition or a symptom of a particular disease or condition, or prevents or delays the onset of a particular disease or condition or a symptom of a particular disease or condition.

The term “patient” means an animal, such as a human, a companion animal, such as a dog, cat and horse, and livestock, such as cattle, swine and sheep. Particularly preferred patients are mammals, including both males and females, with humans being even more preferred.

The term “pharmaceutically acceptable” as used herein means the carrier, vehicle, diluent, excipients and/or salt must be compatible with the other ingredients of the formulation, and not deleterious to the recipient thereof.

The expression “prodrug” refers to a compound that is a drug precursor which, following administration, releases the drug in vivo via some chemical or physiological process (e.g., a prodrug on being brought to the physiological pH or through enzyme action is converted to the desired drug form). Exemplary prodrugs upon cleavage release the corresponding drug compounds.

The expression “pharmaceutically acceptable salt” refers to anionic salts such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine and tromethamine (2-amino-2-hydroxymethyl-1,3-propanediol).

These and other features, aspects and advantages of the present invention will become better understood with reference to the following drawings, description and claims.

### BRIEF DESCRIPTION OF THE DRAWINGS

FIG. 1 is a diffractogram of Form A of (3-(((4-tert-butyl-benzyl)-(pyridine-3-sulfonyl)-amino)-methyl)-phenoxy)-acetic acid sodium salt according to the present invention;

FIG. 2 is a diffractogram of Form B of (3-(((4-tert-butyl-benzyl)-(pyridine-3-sulfonyl)-amino)-methyl)-phenoxy)-acetic acid sodium salt according to the present invention;

FIG. 3 is a diffractogram of Form C of (3-(((4-tert-butyl-benzyl)-(pyridine-3-sulfonyl)-amino)-methyl)-phenoxy)-acetic acid sodium salt according to the present invention;

FIG. 4 is a diffractogram of Form E of (3-(((4-tert-butyl-benzyl)-(pyridine-3-sulfonyl)-amino)-methyl)-phenoxy)-acetic acid sodium salt according to the present invention;

FIG. 5 is a diffractogram of Form F of (3-(((4-tert-butyl-benzyl)-(pyridine-3-sulfonyl)-amino)-methyl)-phenoxy)-acetic acid sodium salt according to the present invention;

FIG. 6 is a diffractogram of Form G of (3-(((4-tert-butyl-benzyl)-(pyridine-3-sulfonyl)-amino)-methyl)-phenoxy)-acetic acid sodium salt according to the present invention;

FIG. 6B is a diffractogram of Form H of (3-(((4-tert-butyl-benzyl)-(pyridine-3-sulfonyl)-amino)-methyl)-phenoxy)-acetic acid sodium salt according to the present invention;

FIG. 6C is a diffractogram of amorphous (3-(((4-tert-butyl-benzyl)-(pyridine-3-sulfonyl)-amino)-methyl)-phenoxy)-acetic acid sodium salt according to the present invention;

FIG. 7 is a solid-state ¹³C nuclear magnetic resonance spectrum of (3-(((4-tert-butyl-benzyl)-(pyridine-3-sulfonyl)-amino)-methyl)-phenoxy)-acetic acid sodium salt, Form A; and

FIG. 8 is a solid-state ¹³C nuclear magnetic resonance spectrum of (3-(((4-tert-butyl-benzyl)-(pyridine-3-sulfonyl)-amino)-methyl)-phenoxy)-acetic acid sodium salt, Form H.

### DETAILED DESCRIPTION OF THE INVENTION

In general, (3-(((4-tert-butyl-benzyl)-(pyridine-3-sulfonyl)-amino)-methyl)-phenoxy)-acetic acid sodium salt may be prepared by methods disclosed in U.S. Pat. No. 6,498,172 B1, the subject matter of which is herein incorporated in its entirety by reference. Alternatively, (3-(((4-tert-butyl-benzyl)-(pyridine-3-sulfonyl)-amino)-methyl)-phenoxy)-acetic acid sodium salt may be prepared by the novel method described in Scheme I, below. Certain processes for the manufacture of specific polymorphs of these compounds are set forth in the experimental section. All starting compounds may be obtained by literature procedures or from general commercial sources, such as Sigma-Aldrich Corporation, St. Louis, Mo.

In the discussions, examples and preparations, the following abbreviations are used 2B EtOH—denatured ethanol, br—broad peaks, ° C.—degree Celsius, d—doublet, DMSO-d₆—deuterated dimethyl sulfoxide, EtOAc—ethyl acetate, equiv—equivalents, g—gram(s), ¹H NMR—proton nuclear magnetic resonance, H₂O—water, HCl—hydrogen chloride, Hz—hertz, iPr₂Net—diisopropyl ethyl amine (Hunig's base), iPrOAc—isopropyl acetate, J—coupling constant (spacing of multiplets), kg—kilogram, L—liter(s), m—multiplet, M—molar, MeCl₂—dichloromethane, mg(s)—milligram(s), min—minute(s), mL—milliliter, mmol—millimole, mp—melting point, MPa—megaPascal, MS—mass spectrum, N—normal, NaHCO₃—sodium hydrogen carbonate, NaOH—sodium hydroxide, psi—pounds per square inch, Pt/C—platinum on carbon, RT—room temperature, s—singlet peak and t—triplet peak. Furthermore, in the following discussions, examples and preparations, reference to Bruker refers to products of Bruker AXS, Inc., Madison, Wis. and reference to Kevex refers to products of Thermo Electron Corporation, Waltham, Mass.

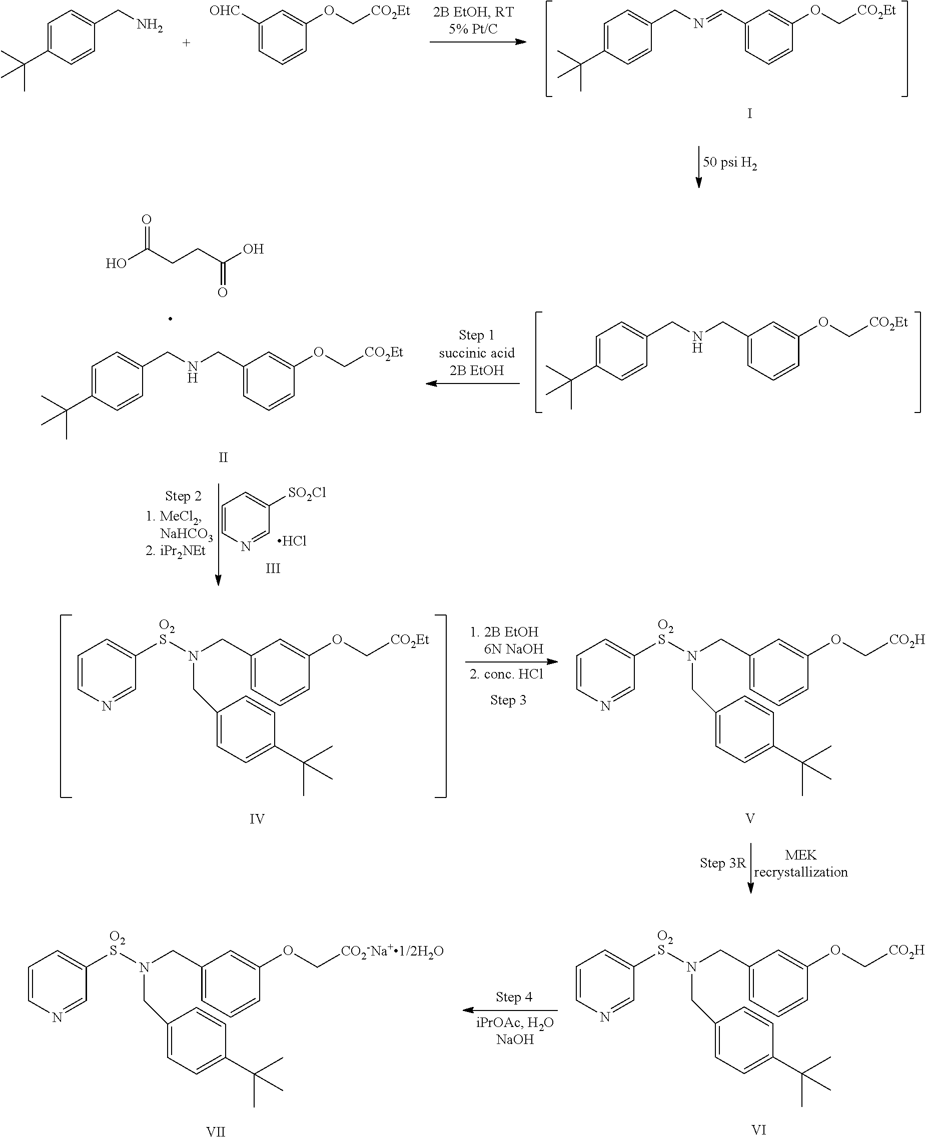

### Step 1: Preparation of {3-[(4-tert-Butyl-benzylamino)-methyl]-phenoxy}-acetic acid ethyl ester succinate (II)

A 500 mL Parr bottle was charged with 2B ethanol (180 mL) and 5% platinum on carbon, 50% water wet (2.00 g). A solution of ethyl-3-formylphenoxyacetate (20.0 g, 96.06 mmol, 1.0 equiv) in 2B ethanol (20 mL) was added, followed by 4-tert-butylbenzyl amine (16.86 mL, 96.06 mmol, 1.0 equiv). The mixture was stirred at room temperature under nitrogen atmosphere for five hours, at which point formation of intermediate of formula I was complete. The reaction vessel was placed under a 50 psi (0.3447 MPa) hydrogen atmosphere and shaken at room temperature for 18 hours. The mixture was filtered through diatomaceous earth, and the filter cake was washed once with 2B ethanol (40 mL). The filtrate was transferred to a one liter flask fitted with an addition funnel and mechanical stirrer. A warm solution of succinic acid (11.34 g, 96.06 mmol, 1.0 equiv) in 2B ethanol (120 mL) was added over 15 minutes. The resulting slurry was stirred at room temperature for 19 hours, then filtered. The solids were washed once with 2B ethanol (50 mL) and dried to afford II (33.10 g, 73% yield). mp=138-139° C. Anal Calcd for C₂₂H₂₉NO₃.C₄H₆O₄: C 65.94; H 7.45; N 2.96. Found: C 65.74; H 7.62; N 2.99. ¹H NMR (400 MHz, CD₃OD): δ 1.28 (t, 3H, J=7.0 Hz), 1.32 (s, 9H), 2.50 (s, 4H), 4.08 (s, 2H), 4.10 (s, 2H), 4.23 (q, 2H, J=7.0 Hz), 4.73 (s, 2H), 6.96-6.98 (m, 1H), 7.04-7.06 (m, 2H), 7.33-7.38 (m, 3H), 7.46-7.48 (m, 2H).

### Steps 2 and 3: Preparation of (3-(((4-tert-butyl-benzyl)-(pyridine-3-sulfonyl)-amino)-methyl)-phenoxy)-acetic acid (V)

Compound II (48.00 g, 101 mmol, 1.0 equiv) was free-based by partitioning between dichloromethane (360 mL) and 1M aqueous sodium bicarbonate (240 mL). The layers were separated and the organic layer washed once with water (240 mL). The layers were separated and N,N-diisopropylethylamine (53.4 mL, 304 mmol, 3.0 equiv) was added to the organic layer. Pyridine-3-sulfonyl chloride hydrochloride (III) (26.4 g, 123 mmol, 1.2 equiv) and dichloromethane (240 mL) were charged to a separate reaction vessel and cooled to 0° C. under nitrogen atmosphere. The free base/N,N-diisopropylethylamine solution was added over 1.8 hours. The reaction mixture was warmed to room temperature and held for 19 hours, at which point formation of intermediate of formula IV ((3-{[(4-tert-Butyl-benzyl)-(pyridine-3-sulfonyl)-amino]-methyl}-phenoxy)-acetic acid ethyl ester) was complete. The reaction mixture was washed with 1N hydrochloric acid (336 mL), then with 1M aqueous sodium bicarbonate (336 mL). 2B ethanol (384 mL) was added to the organic layer, and the mixture was atmospherically distilled until the pot volume was about 400 mL. 6N sodium hydroxide (19.9 mL, 119 mmol, 1.2 equiv) was added and the mixture was held at room temperature for 19 hours. Concentrated hydrochloric acid (11.0 mL, 134 mmol, 1.3 equiv) was added followed by water (192 mL). The slurry granulated 5 hours at room temperature and was filtered. The solids were washed with 50:50 2B ethanol:water (144 mL), water (144 mL), 2B ethanol (144 mL), then dried to afford compound V (34.78 g, 73% yield). mp=159-160° C. Anal Calcd for C₂₅H₂₈N₂O₅S: C 64.08; H 6.02; N 5.98. Found C 64.13; H 6.11; N 5.99. ¹H NMR (400 MHz, DMSO-d₆): δ 1.21 (s, 9H), 4.31 (s, 2H), 4.33 (s, 2H), 4.54 (s, 2H), 6.65-6.69 (m, 1H), 6.71-6.74 (m, 1H), 6.75-6.76 (m, 1H), 7.01 (d, 2H, J=8.1 Hz), 7.13 (t, 1H, J=7.9 Hz), 7.21 (d, 2H, J=8.3 Hz), 7.56-7.59 (m, 1H), 8.16-8.19 (m, 1H), 8.80-8.82 (m, 1H), 8.97-8.98 (m, 1H), 13.04 (br s, 1H).

### Step 3R: Recrystallization of (3-(((4-tert-butyl-benzyl)-(pyridine-3-sulfonyl)-amino)-methyl)-phenoxy)-acetic acid (V)

(3-(((4-tert-Butyl-benzyl)-(pyridine-3-sulfonyl)-amino)-methyl)-phenoxy)-acetic acid (V) (20.0 g, 42.7 mmol, 1.0 equiv) and methyl ethyl ketone (300 mL) were combined and heated to 60° C. until all solids had dissolved. The solution was cooled to 35° C. and filtered through a 0.45 micron nylon filter. The filter was rinsed with methyl ethyl ketone (20 mL). The filtrates were combined and concentrated by atmospheric distillation to a pot volume of approximately 160 mL. The mixture was cooled to room temperature, at which point crystallization commenced. The mixture was stirred 19 hours at room temperature and then filtered. The solids were washed with methyl ethyl ketone (twice with 20 mL, then once with 40 mL) and dried to afford (3-(((4-tert-butyl-benzyl)-(pyridine-3-sulfonyl)-amino)-methyl)-phenoxy)-acetic acid (VI) (15.30 g, 77% yield). mp=159.0-159.5° C. Anal Calcd for C₂₅H₂₈N₂O₅S: C 64.08; H 6.02; N 5.98. Found C 63.89; H 5.82; N 5.91. ¹H NMR (400 MHz, DMSO-d₆): Same as steps 2 and 3 above. The ¹H NMR spectrum also indicated the presence of about 1.4% of methyl ethyl ketone.

### Step 4: Preparation of (3-(((4-tert-butyl-benzyl)-(pyridine-3-sulfonyl)-amino)-methyl)-phenoxy)-acetic acid hemihydrate form A (VII)

(3-(((4-tert-Butyl-benzyl)-(pyridine-3-sulfonyl)-amino)-methyl)-phenoxy)-acetic acid (VI) (20.0 g, 42.7 mmol, 1.0 equiv) and isopropyl acetate (400 mL) were charged to a reaction vessel. 50% sodium hydroxide (3.520 g, 44.0 mmol, 1.03 equiv) was added followed by deionized water (4.165 g). (3-(((4-tert-Butyl-benzyl)-(pyridine-3-sulfonyl)-amino)-methyl)-phenoxy)-acetic acid hemihydrate form A (VII, 200 mg) was added as seed material. The mixture was heated to 70° C. over 30 minutes and held at this temperature for 5 hours. The mixture was cooled to 40° C. over 4 hours, then cooled to 20° C. over 1 hour, granulated 19 hours, and then filtered. The solids were washed twice with 1% aqueous isopropyl acetate (each wash 20 mL), then dried to afford (3-(((4-tert-butyl-benzyl)-(pyridine-3-sulfonyl)-amino)-methyl)-phenoxy)-acetic acid hemihydrate form A (VII, 18.22 g, 85% yield). The hemihydrate has a dehydration temperature at ˜120° C. and not a melting point Anal Calcd for C₂₅H₂₇N₂O₅S.Na.½H₂O: C 61.21; H 5.55; N 5.71. Found C 60.19; H 5.55; N 5.56. ¹H NMR (400 MHz, DMSO-d₆): δ 1.21 (s, 9H), 4.07 (s, 2H), 4.31 (s, 4H), 6.59-6.61 (m, 1H), 6.64 (m, 1H), 6.71-6.74 (m, 1H), 7.01-7.09 (m, 3H), 7.22 (d, 2H, J=8.4 Hz), 7.56-7.59 (m, 1H), 8.14-8.17 (m, 1H), 8.79-8.80 (m, 1H), 8.94 (m, 1H).

Alternatively, the sodium salt formation and recrystallization at Step 4 may be carried out using the procedure shown in the following Scheme II.

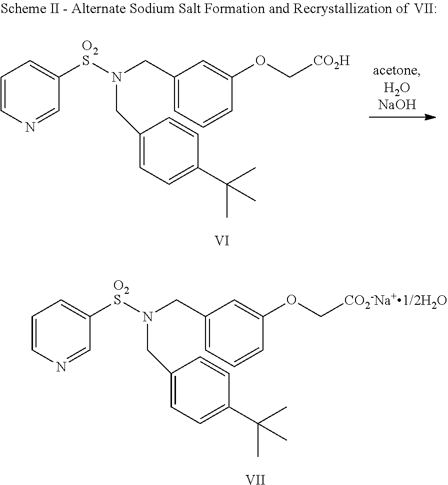

(3-(((4-tert-Butyl-benzyl)-(pyridine-3-sulfonyl)-amino)-methyl)-phenoxy)-acetic acid (VI) (47.7 g) and methanol (500 mL) were combined. 1N sodium hydroxide (1.01 equiv, 103 mL) was added slowly with stirring at room temperature. After stirring 2 hours, the mixture was concentrated and azeotroped five times with acetone (300 mL each time) and twice with dichloromethane (200 mL each time) to give an amorphous foam. To this was added acetone (500 mL) and water (15 mL), and the mixture was heated to 50° C. After 20 minutes the material became very thick, and additional acetone (200 mL) was added. The mixture was cooled to room temperature and filtered. The filter cake was washed with 2-propanol and dried under high vacuum to give 39 g of (3-(((4-tert-Butyl-benzyl)-(pyridine-3-sulfonyl)-amino)-methyl)-phenoxy)-acetic acid hemihydrate form A (VII, 77% yield).

### Powder X-Ray Diffraction

Crystalline Forms A, B, C, E, F, and G of (3-(((4-tert-butyl-benzyl)-(pyridine-3-sulfonyl)-amino)-methyl)-phenoxy)-acetic acid sodium salt were characterized by their X-ray powder diffraction patterns. Thus, the x-ray diffraction patterns of Forms A, B, C, E, F, and G were carried out on a Bruker D5000 diffractometer using copper radiation (wavelength: 1.54056 Å). The tube voltage and amperage were set to 40 kV and 50 mA, respectively. The divergence and scattering slits were set at 1 mm, and the receiving slit was set at 0.6 mm. Diffracted radiation was detected by a Kevex PSI detector. A theta-two theta continuous scan at 2.4°/min (1 sec/0.04° step) from 3.0 to 40°2θ was used. An alumina standard was analyzed to check the instrument alignment. Data were collected and analyzed using Bruker axis software Version 7.0. Samples were prepared by placing them in a quartz holder.

To perform an X-ray diffraction measurement on a Bragg-Brentano instrument like the Bruker system used for measurements reported herein, the sample is typically placed into a holder which has a cavity. The sample powder is pressed by a glass slide or equivalent to ensure a random surface and proper sample height. The sample holder is then placed into the instrument. The incident X-ray beam is directed at the sample, initially at a small angle relative to the plane of the holder, and then moved through an arc that continuously increases the angle between the incident beam and the plane of the holder. Measurement differences associated with such X-ray powder analyses result from a variety of factors including: (a) errors in sample preparation (e.g., sample height), (b) instrument errors (e.g. flat sample errors), (c) calibration errors, (d) operator errors (including those errors present when determining the peak locations), and (e) the nature of the material (e.g. preferred orientation and transparency errors). Calibration errors and sample height errors often result in a shift of all the peaks in the same direction. Small differences in sample height when using a flat holder will lead to large displacements in XRPD peak positions. A systematic study showed that, using a Shimadzu XRD-6000 in the typical Bragg-Brentano configuration, sample height difference of 1 mm lead to peak shifts as high as 1°2θ (Chen et al.; J Pharmaceutical and Biomedical Analysis, 2001; 26, 63). These shifts can be identified from the X-ray Diffractogram and can be eliminated by compensating for the shift (applying a systematic correction factor to all peak position values) or recalibrating the instrument. As mentioned above, it is possible to rectify measurements from the various machines by applying a systematic correction factor to bring the peak positions into agreement. In general, this correction factor will bring the measured peak positions from the Bruker into agreement with the expected peak positions and may be in the range of 0 to 0.2°2θ.

Non-crystalline Form H of (3-(((4-tert-butyl-benzyl)-(pyridine-3-sulfonyl)-amino)-methyl)-phenoxy)-acetic acid sodium salt was characterized by its X-ray powder diffraction pattern. The X-ray powder diffraction pattern for Form H was generated with a Siemens D5000 diffractometer using copper radiation. The instrument was equipped with a line focus X-ray tube. The tube voltage and amperage were set to 38 kV and 38 mA, respectively. The divergence and scattering slits were set at 1 mm, and the receiving slit was set at 0.6 mm. Diffracted Cu K_{α1} radiation (λ=1.54056 Å) was detected using a Kevex PSI detector. A theta two theta continuous scan at 2.4°2θ/min (1 sec/0.04°2θ step) from 3.0 to 40°2θ was used. An alumina standard (NIST standard reference material 1976) was analyzed to check the instrument alignment. Data were collected and analyzed using BRUKER AXS DIFFRAC PLUS software Version 2.0. Samples were prepared for analysis by placing them in a quartz holder. The PXRD peak was manually selected using the maximum peak height.

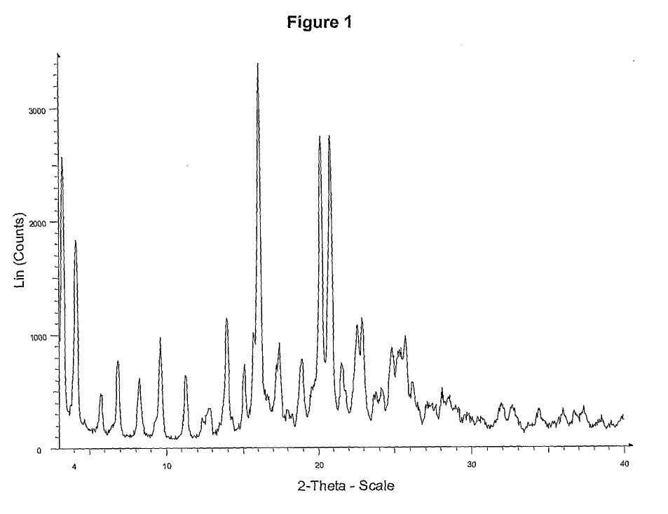

Referring to FIG. 1, there is shown a diffractogram of Form A of (3-(((4-tert-butyl-benzyl)-(pyridine-3-sulfonyl)-amino)-methyl)-phenoxy)-acetic acid sodium salt according to an embodiment of the present invention. Form A of (3-(((4-tert-butyl-benzyl)-(pyridine-3-sulfonyl)-amino)-methyl)-phenoxy)-acetic acid sodium salt is characterized by the following x-ray powder diffraction pattern expressed in terms of the degree 2θ, d-spacings, and relative intensities with a relative intensity of ≧7% measured on a Bruker D5000 diffractometer with CuKα radiation:

TABLE 1
X-ray powder diffraction pattern of sodium salt Form A

| | | Relative* |
|---|---|---|
| Angle | d | Intensity |
| (Degree 2θ) | (Å) | (≧7%) |
| 3.2 | 27.6 | 75.6 |
| 4.1 | 21.6 | 53.9 |
| 5.7 | 15.5 | 14.0 |
| 6.8 | 13.0 | 22.6 |
| 8.2 | 10.8 | 18.0 |
| 9.2 | 9.6 | 7.3 |
| 9.6 | 9.2 | 28.4 |
| 11.2 | 7.9 | 18.6 |
| 12.3 | 7.2 | 7.5 |
| 12.8 | 6.9 | 10.0 |
| 13.9 | 6.4 | 33.3 |
| 15.1 | 5.9 | 21.4 |
| 15.7 | 5.6 | 29.5 |
| 16.1 | 5.5 | 100.0 |
| 17.2 | 5.1 | 21.9 |
| 17.4 | 5.1 | 27.0 |
| 18.0 | 4.9 | 9.7 |
| 18.2 | 4.9 | 8.5 |
| 18.9 | 4.7 | 22.7 |
| 19.6 | 4.5 | 16.0 |
| 20.2 | 4.4 | 80.9 |
| 20.8 | 4.3 | 81.0 |
| 21.6 | 4.1 | 21.6 |
| 22.6 | 3.9 | 31.4 |
| 22.9 | 3.9 | 33.3 |
| 23.7 | 3.7 | 14.2 |
| 24.2 | 3.7 | 15.2 |
| 24.8 | 3.6 | 25.6 |
| 25.3 | 3.5 | 24.9 |
| 25.7 | 3.5 | 28.7 |
| 26.2 | 3.4 | 16.6 |
| 27.3 | 3.3 | 11.3 |
| 27.7 | 3.2 | 10.7 |
| 28.1 | 3.2 | 15.1 |
| 28.5 | 3.1 | 13.3 |
| 32.0 | 2.8 | 11.1 |
| 32.6 | 2.7 | 10.6 |
| 34.4 | 2.6 | 10.1 |
| 36.0 | 2.5 | 9.9 |
| 36.7 | 2.4 | 9.6 |
| 37.4 | 2.4 | 10.9 |

*The relative intensities may change depending on the crystal size and morphology.

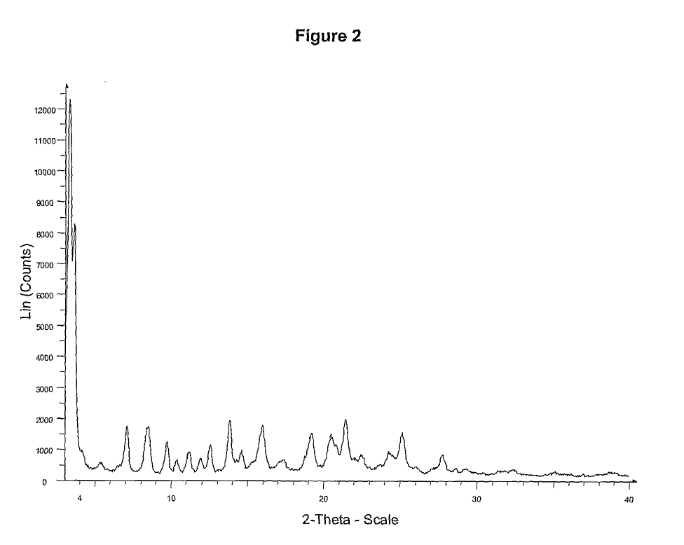

Referring to FIG. 2, there is shown a diffractogram of Form B of (3-(((4-tert-butyl-benzyl)-(pyridine-3-sulfonyl)-amino)-methyl)-phenoxy)-acetic acid sodium salt according to an embodiment of the present invention. Form B of (3-(((4-tert-butyl-benzyl)-(pyridine-3-sulfonyl)-amino)-methyl)-phenoxy)-acetic acid sodium salt is characterized by the following x-ray powder diffraction pattern expressed in terms of the degree 2θ, d-spacings, and relative intensities with a relative intensity of ≧3% measured on a Bruker D5000 diffractometer with CuKα radiation:

TABLE 2
X-ray powder diffraction pattern of sodium salt Form B

| | | Relative* |
|---|---|---|
| Angle | | Intensity |
| (Degree 2θ) | d (Å) | (≧3%) |
| 3.3 | 27.1 | 100.0 |
| 3.6 | 24.7 | 67.3 |
| 4.1 | 21.4 | 7.6 |
| 5.4 | 16.4 | 4.6 |
| 6.5 | 13.5 | 3.6 |
| 7.1 | 12.5 | 14.2 |
| 8.4 | 10.5 | 13.9 |
| 9.7 | 9.1 | 10.2 |
| 10.3 | 8.5 | 5.3 |
| 11.1 | 7.9 | 7.3 |
| 11.9 | 7.4 | 5.7 |
| 12.5 | 7.1 | 9.1 |
| 13.8 | 6.4 | 15.5 |
| 14.6 | 6.1 | 7.7 |
| 15.3 | 5.8 | 4.7 |
| 16.0 | 5.5 | 14.4 |
| 17.3 | 5.1 | 5.3 |
| 19.2 | 4.6 | 12.2 |
| 20.5 | 4.3 | 12.0 |
| 20.8 | 4.3 | 9.4 |
| 21.5 | 4.1 | 15.8 |
| 22.1 | 4.0 | 6.1 |
| 22.5 | 3.9 | 6.6 |
| 23.7 | 3.8 | 4.3 |
| 24.3 | 3.7 | 7.7 |
| 25.2 | 3.5 | 12.5 |
| 26.1 | 3.4 | 3.6 |
| 27.8 | 3.2 | 6.6 |
| 28.7 | 3.1 | 3.2 |
| 29.3 | 3.1 | 3.0 |

*The relative intensities may change depending on the crystal size and morphology.

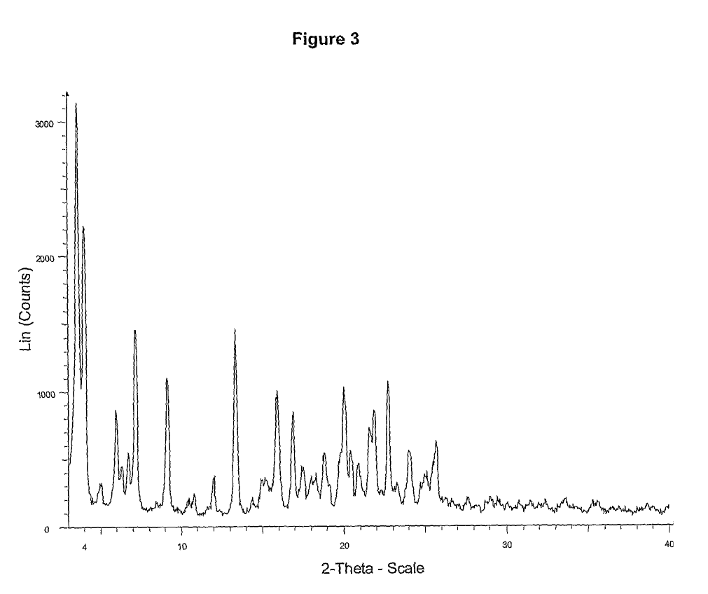

Referring to FIG. 3, there is shown a diffractogram of Form C of (3-(((4-tert-butyl-benzyl)-(pyridine-3-sulfonyl)-amino)-methyl)-phenoxy)-acetic acid sodium salt according to an embodiment of the present invention. Form C of (3-(((4-tert-butyl-benzyl)-(pyridine-3-sulfonyl)-amino)-methyl)-phenoxy)-acetic acid sodium salt is characterized by the following x-ray powder diffraction pattern expressed in terms of the degree 2θ, d-spacings, and relative intensities with a relative intensity of ≧5% measured on a Bruker D5000 diffractometer with CuKα radiation:

TABLE 3
X-ray powder diffraction pattern of sodium salt Form C

| | | Relative* |
|---|---|---|
| Angle | d | Intensity |
| (Degree 2θ) | (Å) | (≧5%) |
| 3.6 | 24.6 | 100.0 |
| 4.0 | 22.1 | 70.8 |
| 5.0 | 17.7 | 10.0 |
| 5.9 | 14.9 | 27.2 |
| 6.3 | 14.1 | 14.0 |
| 6.7 | 13.3 | 17.2 |
| 7.2 | 12.3 | 46.3 |
| 8.4 | 10.5 | 5.7 |
| 9.1 | 9.7 | 34.9 |
| 10.4 | 8.5 | 6.1 |
| 10.7 | 8.2 | 7.5 |
| 11.6 | 7.6 | 5.0 |
| 12.0 | 7.4 | 11.6 |
| 13.3 | 6.6 | 46.2 |
| 14.3 | 6.2 | 6.5 |
| 15.0 | 5.9 | 11.0 |
| 15.2 | 5.8 | 11.3 |
| 15.9 | 5.6 | 31.8 |
| 16.9 | 5.2 | 26.8 |
| 17.5 | 5.1 | 13.6 |
| 18.0 | 4.9 | 11.4 |
| 18.3 | 4.9 | 12.2 |
| 18.8 | 4.7 | 17.1 |
| 20.0 | 4.4 | 32.5 |
| 20.4 | 4.3 | 17.6 |
| 20.9 | 4.3 | 14.5 |
| 21.6 | 4.1 | 22.9 |
| 21.9 | 4.1 | 27.0 |
| 22.7 | 3.9 | 33.9 |
| 23.3 | 3.8 | 10.0 |
| 24.0 | 3.7 | 17.6 |
| 25.0 | 3.6 | 12.1 |
| 25.7 | 3.5 | 19.8 |
| 26.2 | 3.4 | 6.3 |
| 26.7 | 3.3 | 5.7 |
| 27.6 | 3.2 | 6.4 |
| 28.8 | 3.1 | 5.7 |
| 29.1 | 3.1 | 6.5 |
| 29.5 | 3.0 | 6.5 |
| 30.0 | 3.0 | 5.2 |
| 30.8 | 2.9 | 5.7 |
| 31.5 | 2.8 | 5.6 |
| 32.4 | 2.8 | 5.8 |
| 33.6 | 2.7 | 6.2 |
| 35.3 | 2.5 | 5.6 |
| 35.6 | 2.5 | 5.7 |

*The relative intensities may change depending on the crystal size and morphology.

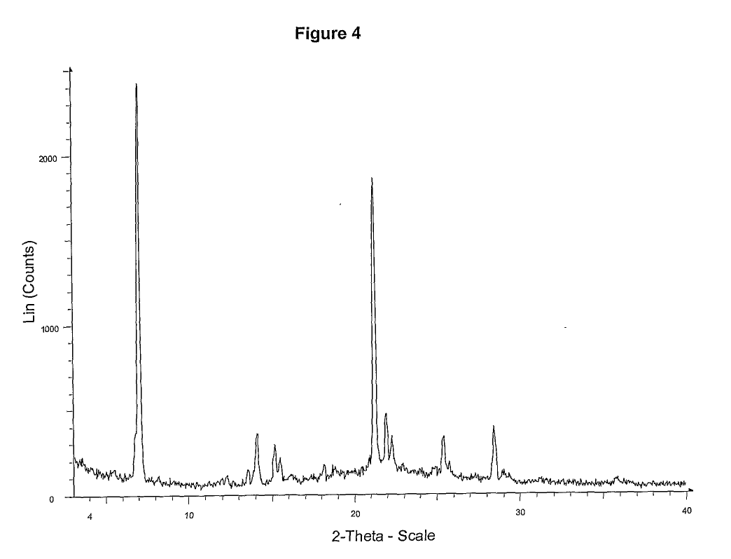

Referring to FIG. 4, there is shown a diffractogram of Form E of (3-(((4-tert-butyl-benzyl)-(pyridine-3-sulfonyl)-amino)-methyl)-phenoxy)-acetic acid sodium salt according to an embodiment of the present invention. Form E of (3-(((4-tert-butyl-benzyl)-(pyridine-3-sulfonyl)-amino)-methyl)-phenoxy)-acetic acid sodium salt is characterized by the following x-ray powder diffraction pattern expressed in terms of the degree 2θ, d-spacings, and relative intensities with a relative intensity of ≧4% measured on a Bruker D5000 diffractometer with CuKα radiation:

TABLE 4
X-ray powder diffraction pattern of sodium salt Form E

| | | Relative* |
|---|---|---|
| Angle | d | Intensity |
| (Degree 2θ) | (Å) | (≧4%) |
| 3.5 | 25.0 | 9.1 |
| 5.4 | 16.2 | 6.1 |
| 7.0 | 12.6 | 100.0 |
| 8.1 | 10.8 | 4.5 |
| 12.0 | 7.4 | 4.0 |
| 12.3 | 7.2 | 4.6 |
| 13.6 | 6.5 | 5.8 |
| 14.1 | 6.3 | 14.6 |
| 15.1 | 5.8 | 11.8 |
| 15.5 | 5.7 | 8.7 |
| 16.2 | 5.5 | 4.7 |
| 18.2 | 4.9 | 7.0 |
| 18.7 | 4.7 | 6.5 |
| 21.2 | 4.2 | 76.6 |
| 21.9 | 4.0 | 19.3 |
| 22.3 | 4.0 | 13.9 |
| 22.9 | 3.9 | 7.1 |
| 24.0 | 3.7 | 6.1 |
| 24.8 | 3.6 | 6.2 |
| 25.4 | 3.5 | 13.8 |
| 25.8 | 3.5 | 7.4 |
| 28.5 | 3.1 | 15.9 |
| 29.0 | 3.1 | 5.5 |
| 29.4 | 3.0 | 4.5 |

*The relative intensities may change depending on the crystal size and morphology.

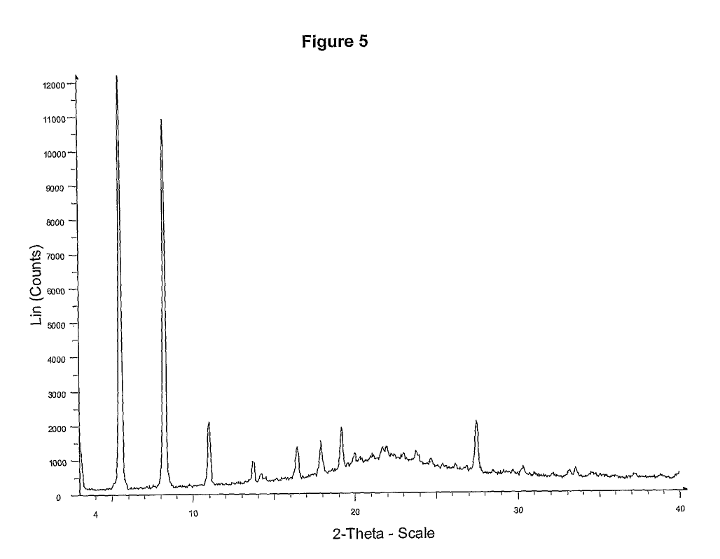

Referring to FIG. 5, there is shown a diffractogram of Form F of (3-(((4-tert-butyl-benzyl)-(pyridine-3-sulfonyl)-amino)-methyl)-phenoxy)-acetic acid sodium salt according to an embodiment of the present invention. Form F of (3-(((4-tert-butyl-benzyl)-(pyridine-3-sulfonyl)-amino)-methyl)-phenoxy)-acetic acid sodium salt is characterized by the following x-ray powder diffraction pattern expressed in terms of the degree 2θ, d-spacings, and relative intensities with a relative intensity of ≧5% measured on a Bruker D5000 diffractometer with CuKα radiation:

TABLE 5
X-ray powder diffraction pattern of sodium salt Form F

| | | Relative* |
|---|---|---|
| Angle | d | Intensity |
| (Degree 2θ) | (Å) | (≧5%) |
| 5.5 | 16.0 | 100.0 |
| 8.2 | 10.7 | 87.2 |
| 11.0 | 8.0 | 16.6 |
| 13.7 | 6.5 | 7.3 |
| 14.2 | 6.2 | 5.0 |
| 16.4 | 5.4 | 10.5 |
| 17.9 | 5.0 | 12.0 |
| 19.2 | 4.6 | 15.1 |
| 20.0 | 4.4 | 9.2 |
| 20.4 | 4.4 | 8.2 |
| 21.1 | 4.2 | 9.0 |
| 21.7 | 4.1 | 10.4 |
| 22.0 | 4.0 | 10.6 |
| 23.0 | 3.9 | 9.0 |
| 23.8 | 3.7 | 9.5 |
| 24.7 | 3.6 | 7.7 |
| 25.4 | 3.5 | 6.5 |
| 26.2 | 3.4 | 6.4 |
| 26.9 | 3.3 | 5.8 |
| 27.5 | 3.2 | 16.5 |
| 30.3 | 2.9 | 5.8 |
| 33.2 | 2.7 | 5.0 |
| 33.6 | 2.7 | 5.4 |

*The relative intensities may change depending on the crystal size and morphology.

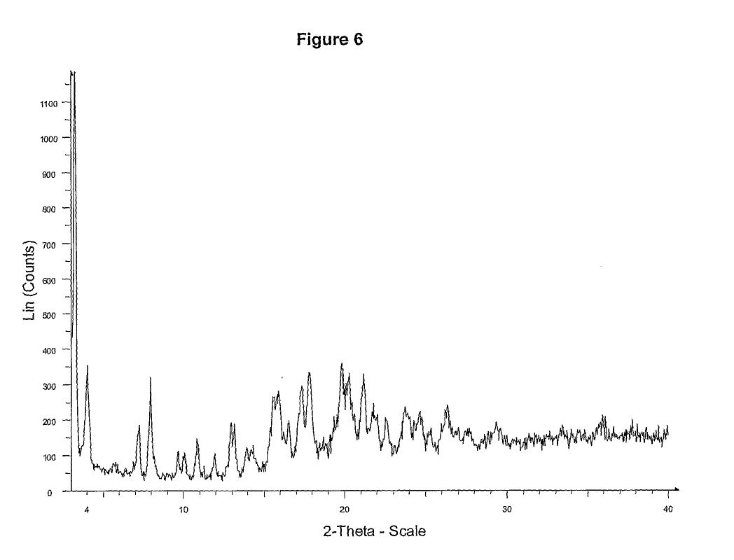

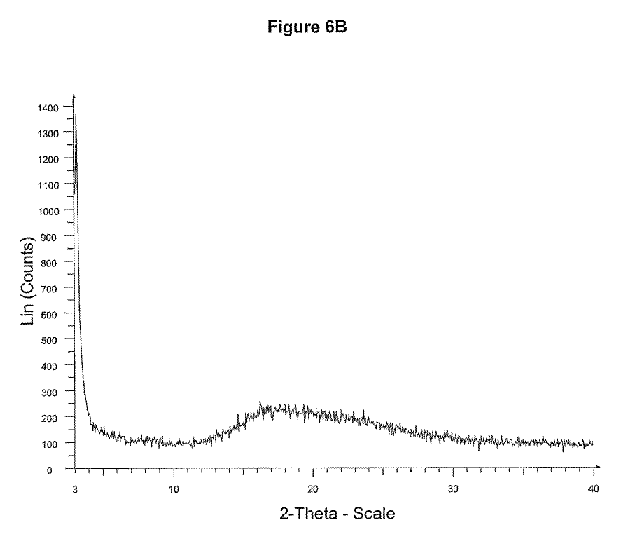

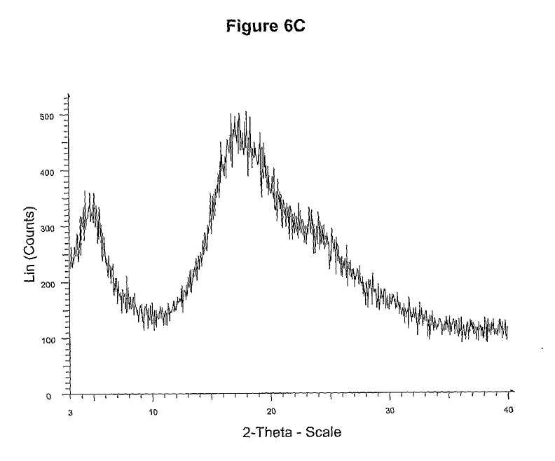

Referring now to FIG. 6, there is shown a diffractogram of Form G of (3-(((4-tert-butyl-benzyl)-(pyridine-3-sulfonyl)-amino)-methyl)-phenoxy)-acetic acid sodium salt according to an embodiment of the present invention. Form G of (3-(((4-tert-butyl-benzyl)-(pyridine-3-sulfonyl)-amino)-methyl)-phenoxy)-acetic acid sodium salt is characterized by the following x-ray powder diffraction pattern expressed in terms of the degree 2θ, d-spacings, and relative intensities with a relative intensity of ≧6% measured on a Bruker D5000 diffractometer with CuKα radiation:

TABLE 6
X-ray powder diffraction pattern of sodium salt Form G

| | | Relative* |
|---|---|---|
| Angle | d | Intensity |
| (Degree 2θ) | (Å) | (≧6%) |
| 3.2 | 27.8 | 100.0 |
| 4.0 | 22.3 | 28.6 |
| 5.6 | 15.7 | 6.2 |
| 7.2 | 12.3 | 14.9 |
| 7.9 | 11.2 | 25.9 |
| 9.6 | 9.2 | 8.8 |
| 10.0 | 8.8 | 8.5 |
| 10.8 | 8.2 | 11.7 |
| 11.9 | 7.4 | 8.4 |
| 12.9 | 6.9 | 15.5 |
| 13.1 | 6.7 | 15.1 |
| 13.9 | 6.4 | 9.8 |
| 14.3 | 6.2 | 9.3 |
| 15.6 | 5.7 | 21.5 |
| 15.8 | 5.6 | 22.7 |
| 16.5 | 5.4 | 15.8 |
| 17.3 | 5.1 | 23.7 |
| 17.8 | 5.0 | 27.0 |
| 19.8 | 4.5 | 29.1 |
| 20.2 | 4.4 | 26.7 |
| 21.2 | 4.2 | 26.5 |
| 21.7 | 4.1 | 19.8 |
| 22.0 | 4.0 | 17.3 |
| 22.6 | 3.9 | 16.1 |
| 23.8 | 3.7 | 18.8 |
| 24.6 | 3.6 | 18.0 |
| 25.3 | 3.5 | 13.5 |
| 26.3 | 3.4 | 19.4 |
| 29.4 | 3.0 | 15.5 |

*The relative intensities may change depending on the crystal size and morphology.

In summary, Table 7 lists the degree 2θ and relative intensities of the diffraction lines in the sample for crystalline Forms A, B, C, E, F, and G of (3-(((4-tert-butyl-benzyl)-(pyridine-3-sulfonyl)-amino)-methyl)-phenoxy)-acetic acid sodium salt:

TABLE 7
PEAK LOCATIONS AND INTENSITIES OF DIFFRACTION LINES
for forms A, B, C, E, F, and G OF (3-(((4-tert-butyl-benzyl)-(pyridine-3-
sulfonyl)-amino)-methyl)-phenoxy)-acetic acid sodium salt

| FORM A | | FORM B | | FORM C | | FORM E | | FORM F | | FORM G | |
|---|---|---|---|---|---|---|---|---|---|---|---|
| Angle | Relative | Angle | Relative | Angle | Relative | Angle | Relative | Angle | Relative | Angle | Relative |
| (Degree | Intensity | (Degree | Intensity | (Degree | Intensity | (Degree | Intensity | (Degree | Intensity | (Degree | Intensity |
| 2θ) | (≧7%) | 2θ) | (≧3%) | 2θ) | (≧5%) | 2θ) | (≧4%) | 2θ) | (≧5%) | 2θ) | (≧6%) |
| 3.2 | 75.6 | 3.3 | 100.0 | 3.6 | 100.0 | 3.5 | 9.1 | 5.5 | 100.0 | 3.2 | 100.0 |
| 4.1 | 53.9 | 3.6 | 67.3 | 4.0 | 70.8 | 5.4 | 6.1 | 8.2 | 87.2 | 4.0 | 28.6 |
| 5.7 | 14.0 | 4.1 | 7.6 | 5.0 | 10.0 | 7.0 | 100.0 | 11.0 | 16.6 | 5.6 | 6.2 |
| 6.8 | 22.6 | 5.4 | 4.6 | 5.9 | 27.2 | 8.1 | 4.5 | 13.7 | 7.3 | 7.2 | 14.9 |
| 8.2 | 18.0 | 6.5 | 3.6 | 6.3 | 14.0 | 12.0 | 4.0 | 14.2 | 5.0 | 7.9 | 25.9 |
| 9.2 | 7.3 | 7.1 | 14.2 | 6.7 | 17.2 | 12.3 | 4.6 | 16.4 | 10.5 | 9.6 | 8.8 |
| 9.6 | 28.4 | 8.4 | 13.9 | 7.2 | 46.3 | 13.6 | 5.8 | 17.9 | 12.0 | 10.0 | 8.5 |
| 11.2 | 18.6 | 9.7 | 10.2 | 8.4 | 5.7 | 14.1 | 14.6 | 19.2 | 15.1 | 10.8 | 11.7 |
| 12.3 | 7.5 | 10.3 | 5.3 | 9.1 | 34.9 | 15.1 | 11.8 | 20.0 | 9.2 | 11.9 | 8.4 |
| 12.8 | 10.0 | 11.1 | 7.3 | 10.4 | 6.1 | 15.5 | 8.7 | 20.4 | 8.2 | 12.9 | 15.5 |
| 13.9 | 33.3 | 11.9 | 5.7 | 10.7 | 7.5 | 16.2 | 4.7 | 21.1 | 9.0 | 13.1 | 15.1 |
| 15.1 | 21.4 | 12.5 | 9.1 | 11.6 | 5.0 | 18.2 | 7.0 | 21.7 | 10.4 | 13.9 | 9.8 |
| 15.7 | 29.5 | 13.8 | 15.5 | 12.0 | 11.6 | 18.7 | 6.5 | 22.0 | 10.6 | 14.3 | 9.3 |
| 16.1 | 100.0 | 14.6 | 7.7 | 13.3 | 46.2 | 21.2 | 76.6 | 23.0 | 9.0 | 15.6 | 21.5 |
| 17.2 | 21.9 | 15.3 | 4.7 | 14.3 | 6.5 | 21.9 | 19.3 | 23.8 | 9.5 | 15.8 | 22.7 |
| 17.4 | 27.0 | 16.0 | 14.4 | 15.0 | 11.0 | 22.3 | 13.9 | 24.7 | 7.7 | 16.5 | 15.8 |
| 18.0 | 9.7 | 17.3 | 5.3 | 15.2 | 11.3 | 22.9 | 7.1 | 25.4 | 6.5 | 17.3 | 23.7 |
| 18.2 | 8.5 | 19.2 | 12.2 | 15.9 | 31.8 | 24.0 | 6.1 | 26.2 | 6.4 | 17.8 | 27.0 |
| 18.9 | 22.7 | 20.5 | 12.0 | 16.9 | 26.8 | 24.8 | 6.2 | 26.9 | 5.8 | 19.8 | 29.1 |
| 19.6 | 16.0 | 20.8 | 9.4 | 17.5 | 13.6 | 25.4 | 13.8 | 27.5 | 16.5 | 20.2 | 26.7 |
| 20.2 | 80.9 | 21.5 | 15.8 | 18.0 | 11.4 | 25.8 | 7.4 | 30.3 | 5.8 | 21.2 | 26.5 |
| 20.8 | 81.0 | 22.1 | 6.1 | 18.3 | 12.2 | 28.5 | 15.9 | 33.2 | 5.0 | 21.7 | 19.8 |
| 21.6 | 21.6 | 22.5 | 6.6 | 18.8 | 17.1 | 29.0 | 5.5 | 33.6 | 5.4 | 22.0 | 17.3 |
| 22.6 | 31.4 | 23.7 | 4.3 | 20.0 | 32.5 | 29.4 | 4.5 | | | 22.6 | 16.1 |
| 22.9 | 33.3 | 24.3 | 7.7 | 20.4 | 17.6 | | | | | 23.8 | 18.8 |
| 23.7 | 14.2 | 25.2 | 12.5 | 20.9 | 14.5 | | | | | 24.6 | 18.0 |
| 24.2 | 15.2 | 26.1 | 3.6 | 21.6 | 22.9 | | | | | 25.3 | 13.5 |
| 24.8 | 25.6 | 27.8 | 6.6 | 21.9 | 27.0 | | | | | 26.3 | 19.4 |
| 25.3 | 24.9 | 28.7 | 3.2 | 22.7 | 33.9 | | | | | 29.4 | 15.5 |
| 25.7 | 28.7 | 29.3 | 3.0 | 23.3 | 10.0 | | | | | | |
| 26.2 | 16.6 | | | 24.0 | 17.6 | | | | | | |
| 27.3 | 11.3 | | | 25.0 | 12.1 | | | | | | |
| 27.7 | 10.7 | | | 25.7 | 19.8 | | | | | | |
| 28.1 | 15.1 | | | 26.2 | 6.3 | | | | | | |
| 28.5 | 13.3 | | | 26.7 | 5.7 | | | | | | |
| 32.0 | 11.1 | | | 27.6 | 6.4 | | | | | | |
| 32.6 | 10.6 | | | 28.8 | 5.7 | | | | | | |
| 34.4 | 10.1 | | | 29.1 | 6.5 | | | | | | |
| 36.0 | 9.9 | | | 29.5 | 6.5 | | | | | | |
| 36.7 | 9.6 | | | 30.0 | 5.2 | | | | | | |
| 37.4 | 10.9 | | | 30.8 | 5.7 | | | | | | |
| | | | | 31.5 | 5.6 | | | | | | |
| | | | | 32.4 | 5.8 | | | | | | |
| | | | | 33.6 | 6.2 | | | | | | |
| | | | | 35.3 | 5.6 | | | | | | |
| | | | | 35.6 | 5.7 | | | | | | |

Because only six crystalline forms of (3-(((4-tert-butyl-benzyl)-(pyridine-3-sulfonyl)-amino)-methyl)-phenoxy)-acetic acid sodium salt are known, each form can be identified and distinguished from the other crystalline forms by either a single x-ray powder diffraction line, a combination of lines or a pattern that is different from the x-ray powder diffraction of the other forms.

For example, Table 8 lists unique peaks, as well as combinations of 2θ peaks for Forms A, B, C, E, F, and G of (3-(((4-tert-butyl-benzyl)-(pyridine-3-sulfonyl)-amino)-methyl)-phenoxy)-acetic acid sodium salt, i.e., a set of x-ray diffraction lines that are unique to each form.

TABLE 8
UNIQUE PEAKS AND COMBINATIONS OF 2θ PEAKS
for forms A, B, C, E, F, and G OF (3-(((4-tert-butyl-benzyl)-(pyridine-
3-sulfonyl)-amino)-methyl)-phenoxy)-acetic acid sodium salt

| FORM A | FORM B | FORM C | FORM E | FORM F | FORM G |
|---|---|---|---|---|---|
| (Degree | (Degree | (Degree | (Degree | (Degree | (Degree |
| 2θ) | 2θ) | 2θ) | 2θ) | 2θ) | 2θ) |
| 3.2 | 3.3 | 3.6 | 7.0 | 5.5 | 3.2 |
| 4.1 | 3.6 | 4.0 | 14.1 | 8.2 | 4.0 |
| 16.1 | 7.1 | 5.9 | 21.2 | 11.0 | 7.9 |
| 20.2 | 8.4 | 13.3 | 22.3 | 19.2 | 10.0 |
| 20.8 | 13.8 | 16.9 | 28.5 | 27.5 | 13.1 |

Referring now to FIG. 6B, there is shown a diffractogram of non-crystalline Form H of (3-(((4-tert-butyl-benzyl)-(pyridine-3-sulfonyl)-amino)-methyl)-phenoxy)-acetic acid sodium salt according to an embodiment of the present invention. Form H of (3-(((4-tert-butyl-benzyl)-(pyridine-3-sulfonyl)-amino)-methyl)-phenoxy)-acetic acid sodium salt is characterized by the following x-ray powder diffraction pattern expressed in terms of the degree 2θ, d-spacings, and relative intensities with a relative intensity of ≧6% measured on a Bruker D5000 diffractometer with CuKα radiation:

| Angle | d value | |
|---|---|---|
| 2-Theta ° | Angstrom | Intensity % |
| 3.1 | 28.1 | 100.0 |

Referring now to FIG. 6C, there is shown a diffractogram of amorphous Form I of (3-(((4-tert-butyl-benzyl)-(pyridine-3-sulfonyl)-amino)-methyl)-phenoxy)-acetic acid sodium salt according to an embodiment of the present invention. Form I of (3-(((4-tert-butyl-benzyl)-(pyridine-3-sulfonyl)-amino)-methyl)-phenoxy)-acetic acid sodium salt is characterized by the x-ray powder diffraction pattern as shown in the figure.

### Solid State Nuclear Magnetic Resonance

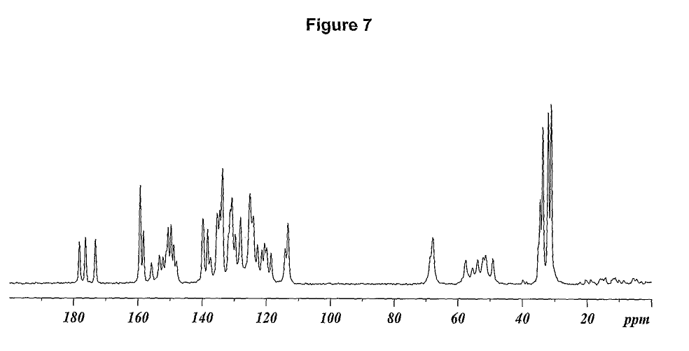

Referring now to FIG. 7, Form A of (3-(((4-tert-butyl-benzyl)-(pyridine-3-sulfonyl)-amino)-methyl)-phenoxy)-acetic acid sodium salt may also be characterized by its solid-state nuclear magnetic resonance spectra (SSNMR). Thus, the solid-state nuclear magnetic resonance spectrum of Form A was carried out on Bruker-Biospin (Buker-Biospin, Billerica, Mass.) Avance DSX 500 MHz NMR spectrometer.

### ¹³C SSNMR

Approximately 70 mg of compound were tightly packed into a 4 mm ZrO spinner for the sample analyzed. The one-dimensional ¹³C spectra were collected at ambient pressure using ¹H-¹³C cross-polarization magic angle spinning (CPMAS) at 293 K on a Bruker 4 mm BL CPMAS probe positioned into a wide-bore Bruker-Biospin Avance DSX 500 MHz NMR spectrometer. The sample was spun at 15.0 kHz corresponding to the maximum specified spinning speed for the 4 mm spinners. The fast spinning speed minimized the intensities of the spinning side bands. To optimize the signal sensitivity, the cross-polarization contact time was adjusted to 2.0 ms, and the decoupling power was set to 85 kHz. The carbon spectra were acquired with 2,820 scans with a recycle delay of 3 seconds. They were referenced using an external sample of adamantane, setting its upfield resonance to 29.5 ppm.

Form A of (3-(((4-tert-butyl-benzyl)-(pyridine-3-sulfonyl)-amino)-methyl)-phenoxy)-acetic acid sodium salt was characterized by the following solid-state ¹³C nuclear magnetic resonance (SSNMR) spectrum wherein chemical shift is expressed in parts per million (ppm):

TABLE 9
Solid-state ¹³C nuclear magnetic resonance
spectrum of Form A

| | ¹³C shift |
|---|---|
| # | [ppm] |
| 1 | 178.2 |
| 2 | 176.3 |
| 3 | 173.2 |
| 4 | 159.3 |
| 5 | 158.3 |
| 6 | 155.7 |
| 7 | 153.3 |
| 8 | 152.2 |
| 9 | 150.6 |
| 10 | 149.6 |
| 11 | 148.8 |
| 12 | 148.0 |
| 13 | 139.8 |
| 14 | 138.3 |
| 15 | 137.4 |
| 16 | 135.3 |
| 17 | 134.6 |
| 18 | 133.7 |
| 19 | 131.2 |
| 20 | 130.7 |
| 21 | 129.6 |
| 22 | 128.0 |
| 23 | 125.1 |
| 24 | 124.0 |
| 25 | 122.7 |
| 26 | 121.3 |
| 27 | 120.5 |
| 28 | 119.9 |
| 29 | 118.4 |
| 30 | 114.1 |
| 31 | 113.2 |
| 32 | 67.8 |
| 33 | 57.5 |
| 34 | 55.4 |
| 35 | 53.9 |
| 36 | 52.1 |
| 37 | 51.3 |
| 38 | 49.1 |
| 39 | 34.5 |
| 40 | 33.7 |
| 41 | 32.1 |
| 42 | 31.2 |

*Values in ppm with respect to trimethylsilane (TMS) at 0 ppm; referenced using an external sample of adamantane, setting is upfield resonance to 29.5 ppm.

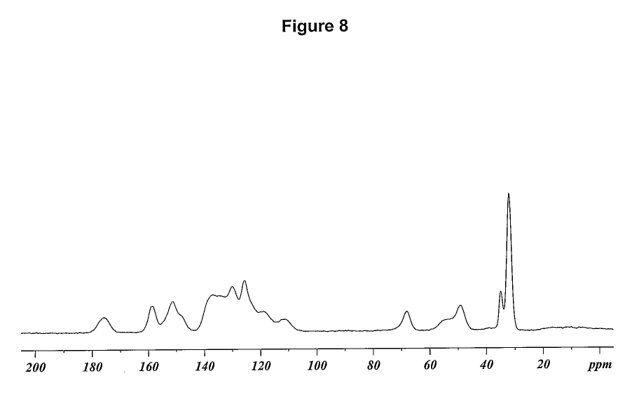

Referring now to FIG. 8, Form H of (3-(((4-tert-butyl-benzyl)-(pyridine-3-sulfonyl)-amino)-methyl)-phenoxy)-acetic acid sodium salt may also be characterized by its solid-state nuclear magnetic resonance spectra (SSNMR). Thus, the solid-state nuclear magnetic resonance spectrum of Form A was carried out on Bruker-Biospin (Buker-Biospin, Billerica, Mass.) Avance DSX 500 MHz NMR spectrometer.

### ¹³CSSNMR Method for Form H

Approximately 70 mg of compound were tightly packed into a 4 mm ZrO₂ rotor. The one-dimensional ¹³C spectra were collected at ambient pressure using ¹H-¹³C cross-polarization magic angle spinning (CPMAS) at 294 K on a Bruker 4 mm BL CPMAS probe positioned into a wide-bore Bruker-Biospin Avance DSX 500 MHz NMR spectrometer. The sample was spun at 15.0 kHz corresponding to the maximum specified spinning speed for the 4 mm rotors. The fast spinning speed minimized the intensities of the spinning side bands. To optimize the signal sensitivity, the cross-polarization contact time was adjusted to 2.0 ms, and the decoupling power was set to 85 kHz. The carbon spectra were acquired with 5400 scans with a recycle delay of 5 seconds. They were referenced using an external sample of adamantane, setting its upfield resonance to 29.5 ppm.

TABLE 10
Solid-state ¹³C nuclear magnetic resonance spectrum
of Form H

| ¹³C Chemical | |
|---|---|
| Shifts | |
| [ppm] | Intensity |
| 175.8 | 1.0 |
| 158.6 | 2.0 |
| 151.2 | 2.4 |
| 148.7* | 1.2 |
| 136.9 | 3.0 |
| 130.0 | 3.7 |
| 125.7 | 4.3 |
| 118.8 | 1.5 |
| 111.0 | 0.8 |
| 68.0 | 1.4 |
| 53.9* | 0.7 |
| 49.1 | 1.9 |
| 34.9 | 3.2 |
| 31.9 | 12.0 |

*Peak shoulder

Crystalline forms of (3-(((4-tert-butyl-benzyl)-(pyridine-3-sulfonyl)-amino)-methyl)-phenoxy)-acetic acid sodium salt of the present invention or the non-crystalline form or amorphous form may exist in anhydrous forms as well as hydrated and solvated forms. In general, the hydrated forms are equivalent to unhydrated forms and are intended to be encompassed within the scope of the present invention. Crystalline Forms A, B, and C of (3-(((4-tert-butyl-benzyl)-(pyridine-3-sulfonyl)-amino)-methyl)-phenoxy)-acetic acid sodium salt preferably occur as hydrates while Forms E, F and G of (3-(((4-tert-butyl-benzyl)-(pyridine-3-sulfonyl)-amino)-methyl)-phenoxy)-acetic acid sodium salt preferably occur as anhydrous forms.

Crystalline forms or the non-crystalline form or amorphous form of (3-(((4-tert-butyl-benzyl)-(pyridine-3-sulfonyl)-amino)-methyl)-phenoxy)-acetic acid sodium salt of the present invention, regardless of the extent of hydration and/or solvation having equivalent x-ray powder diffractograms, or solid-state NMR spectra, are within the scope of the present invention.

The following nonlimiting examples illustrate preferred methods for preparing the compounds of the invention.

### Examples

### Form A: (3-(((4-tert-butyl-benzyl)-(pyridine-3-sulfonyl)-amino)-methyl)-phenoxy)-acetic acid sodium salt

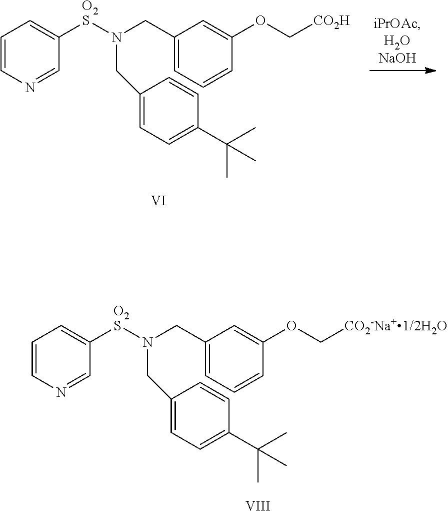

Procedure: A 500 mL jacketed reactor was charged with isopropyl acetate (400 mL), 50% aqueous sodium hydroxide (3.520 g) and water (4.165 g). (3-(((4-tert-butyl-benzyl)-(pyridine-3-sulfonyl)-amino)-methyl)-phenoxy)-acetic acid (VI) was charged to the mixture (20.0 g), followed by Form A of (3-(((4-tert-butyl-benzyl)-(pyridine-3-sulfonyl)-amino)-methyl)-phenoxy)-acetic acid sodium salt (which is prepared by, for example, the process of Scheme II, above) as seed (200 mg). The mixture was heated to 70° C. over 30 minutes and stirred at this temperature for five hours. The mixture was cooled to 40° C. over four hours, then cooled to 20° C. over one hour. The slurry then granulated at 20° C. for 12 hours. The slurry was filtered through a Buchner funnel (filter paper). The filter cake was washed twice with 20 mL of 1% aqueous isopropyl acetate, then dried in a vacuum oven overnight at 40-45° C. with a nitrogen bleed to give 16.84 g of (3-(((4-tert-butyl-benzyl)-(pyridine-3-sulfonyl)-amino)-methyl)-phenoxy)-acetic acid sodium salt hemihydrate Form A (VIII, 79% yield).

### Form B: (3-(((4-tert-butyl-benzyl)-(pyridine-3-sulfonyl)-amino)-methyl)-phenoxy)-acetic acid sodium salt

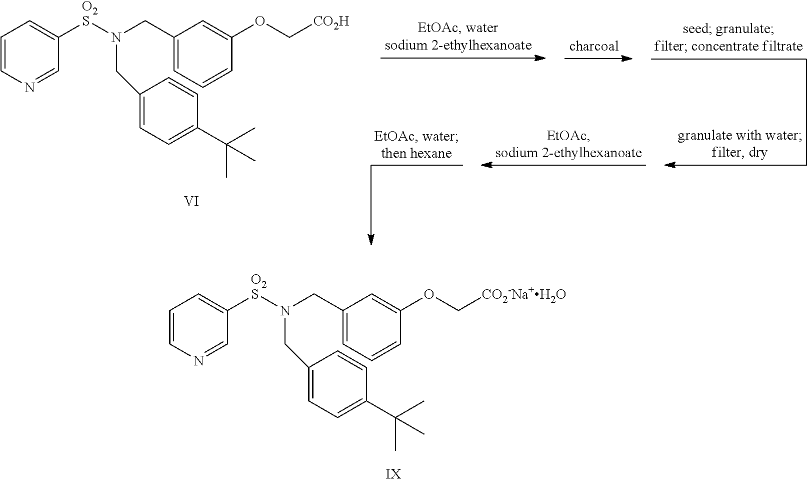

Procedure: (3-(((4-tert-butyl-benzyl)-(pyridine-3-sulfonyl)-amino)-methyl)-phenoxy)-acetic acid (VI) (4.98 g), ethyl acetate (59.8 mL) and water (1.3 mL) were combined and heated to 55° C. A solution of sodium 2-ethylhexanoate (97%, 2.025 g) in ethyl acetate (20 mL) and water (0.44 mL) was added, resulting in a clear solution. The solution was treated with activated charcoal for 15 min at 55°-60° C. and filtered through diatomaceous earth. The filter cake was washed once with 5 mL of ethyl acetate/2.2% water. The filtrate was 40° C. 50 mg of (3-(((4-tert-butyl-benzyl)-(pyridine-3-sulfonyl)-amino)-methyl)-phenoxy)-acetic acid sodium salt (VIII) were added as seed, and the mixture was allowed to cool to room temperature and stirred for 18 hours. The slurry was filtered to give 3.1 g of (3-(((4-tert-butyl-benzyl)-(pyridine-3-sulfonyl)-amino)-methyl)-phenoxy)-acetic acid sodium salt.

The filtrate was concentrated to 20 mL, seeded with (3-(((4-tert-butyl-benzyl)-(pyridine-3-sulfonyl)-amino)-methyl)-phenoxy)-acetic acid sodium salt (VIII), stirred 3 hours at room temperature and filtered to give 0.1 g of (3-(((4-tert-butyl-benzyl)-(pyridine-3-sulfonyl)-amino)-methyl)-phenoxy)-acetic acid sodium salt.

The second filtrate was concentrated to dryness, granulated in 40 mL water at room temperature, filtered and air dried to give 2.4 g of solids. These were combined with 30 mL ethyl acetate and a solution of sodium 2-ethylhexanoate (97%, 1 g) in 10 mL ethyl acetate. The mixture was stirred at room temperature for 18 hours, filtered and dried to give a non-crystalline solid. This material was dissolved in 40 mL ethyl acetate and 1 mL water, and stirred at room temperature to 18 hours. Hexane was added until the haze point was reached, then the mixture was allowed to stir 24 hours. A thick precipitate formed, which was filtered, washed with dry ethyl acetate, and dried to give 1.27 g of (3-(((4-tert-butyl-benzyl)-(pyridine-3-sulfonyl)-amino)-methyl)-phenoxy)-acetic acid sodium salt monohydrate Form B (IX).

### Form C: (3-(((4-tert-butyl-benzyl)-(pyridine-3-sulfonyl)-amino)-methyl)-phenoxy)-acetic acid sodium salt

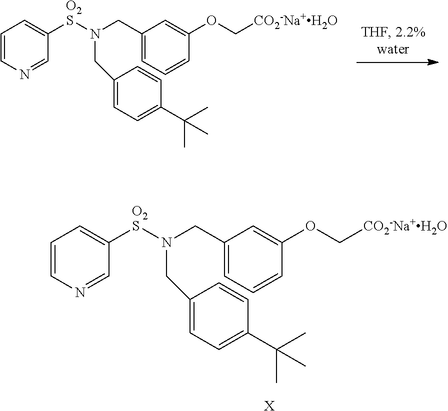

Procedure: (3-(((4-tert-butyl-benzyl)-(pyridine-3-sulfonyl)-amino)-methyl)-phenoxy)-acetic acid sodium salt (501 mg) and 5.0 mL of wet THF (2.2% water) were combined and heated to 50°-55° C. over one hour under nitrogen atmosphere. The suspension was seeded with 4 mg of (3-(((4-tert-butyl-benzyl)-(pyridine-3-sulfonyl)-amino)-methyl)-phenoxy)-acetic acid sodium salt and then allowed to cool to room temperature and allowed to stir for 66 hours. The solvent had completely evaporated, leaving behind a white solid. An additional 5 mL of wet THF (2.2% water) were added and the mixture stirred for 5 hours. The solids were filtered and dried under vacuum at 45°-50° C. to give 333 mg of (3-(((4-tert-butyl-benzyl)-(pyridine-3-sulfonyl)-amino)-methyl)-phenoxy)-acetic acid sodium salt monohydrate Form C (X, 65.3% yield).

### Forms E and F: (3-(((4-tert-butyl-benzyl)-(pyridine-3-sulfonyl)-amino)-methyl)-phenoxy)-acetic acid sodium salt

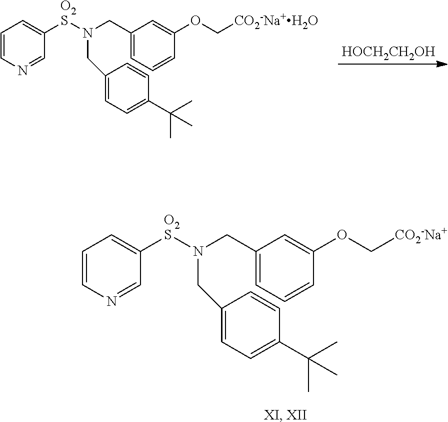

Procedure: (3-(((4-tert-butyl-benzyl)-(pyridine-3-sulfonyl)-amino)-methyl)-phenoxy)-acetic acid sodium salt was dissolved in ethylene glycol, and the solution was allowed to slowly evaporate at room temperature. After 1 week, fine needles formed, which were filtered, air dried and characterized as (3-(((4-tert-butyl-benzyl)-(pyridine-3-sulfonyl)-amino)-methyl)-phenoxy)-acetic acid sodium salt anhydrous Form E (XI).

The filtrate was evaporated to a low volume and filtered. The isolated product was characterized as (3-(((4-tert-butyl-benzyl)-(pyridine-3-sulfonyl)-amino)-methyl)-phenoxy)-acetic acid sodium salt anhydrous form F (XII).

### Form G: (3-(((4-tert-butyl-benzyl)-(pyridine-3-sulfonyl)-amino)-methyl)-phenoxy)-acetic acid sodium salt

Procedure: (3-(((4-tert-butyl-benzyl)-(pyridine-3-sulfonyl)-amino)-methyl)-phenoxy)-acetic acid sodium salt (50 mg) was heated to 155° C. over one hour to produce (3-(((4-tert-butyl-benzyl)-(pyridine-3-sulfonyl)-amino)-methyl)-phenoxy)-acetic acid sodium salt Form G.

In addition to the six crystalline forms (Forms A-G) of (3-(((4-tert-butyl-benzyl)-(pyridine-3-sulfonyl)-amino)-methyl)-phenoxy)-acetic acid sodium salt that have been identified and described herein a non-crystalline form of (3-(((4-tert-butyl-benzyl)-(pyridine-3-sulfonyl)-amino)-methyl)-phenoxy)-acetic acid sodium salt, designated Form H, has also been identified.

### Preparation of non-crystalline (3-(((4-tert-butyl-benzyl)-(pyridine-3-sulfonyl)-amino)-methyl)-phenoxy)-acetic acid sodium salt (Form H)

The following two methods can be used to prepare non-crystalline Form H of (3-(((4-tert-butyl-benzyl)-(pyridine-3-sulfonyl)-amino)-methyl)-phenoxy)-acetic acid sodium salt.

Form H, Method 1. A solution of 20-100 mg/mL of (3-(((4-tert-butyl-benzyl)-(pyridine-3-sulfonyl)-amino)-methyl)-phenoxy)-acetic acid sodium salt was made by dissolving the drug substance in Water for Injections. The solution was filtered with a 0.22μ sterile filter. The solution was filled into vials/syringes. The vials/syringes were frozen at −45° C. and held at that temperature for 2 hours. The freeze-drying was completed by primary drying at (−20 to 25° C.) at 150 mτ, followed by secondary drying at (20 to 30° C.). The resulting solid is stored.

Form H, Method 2. Dissolve 1 gram of (3-(((4-tert-butyl-benzyl)-(pyridine-3-sulfonyl)-amino)-methyl)-phenoxy)-acetic acid sodium salt in 10 mL water for injection to make 100 mg/mL solution. The solution is filtered with a 0.22μ sterile filter. Aliquots of 0.5 mL solution are filled into glass vials. The vials are loaded into freeze-dryer. The shelf temperature is ramped to −45° C. at the rate of 0.5° C./minute. Hold the shelf temperature at −45° C. for 2 hours. Apply vacuum at 150 mτ. Ramp the shelf temperature to −20° C. at the rate of 0.5° C./minute. Hold the shelf temperature at −10° C. for 20 hours. Ramp the shelf temperature to 30° C. at the rate of 0.5° C./minute. Hold at 30° C. for 10 hours. Maintain the vacuum at 150 mτ throughout the freeze-drying process.

In addition to the six crystalline forms (Forms A-G) of (3-(((4-tert-butyl-benzyl)-(pyridine-3-sulfonyl)-amino)-methyl)-phenoxy)-acetic acid sodium salt and non-crystalline Form H that have been identified and described herein an amorphous form of (3-(((4-tert-butyl-benzyl)-(pyridine-3-sulfonyl)-amino)-methyl)-phenoxy)-acetic acid sodium salt, designated Form I, has also been identified.

The following two methods can be used to prepare amorphous Form I of (3-(((4-tert-butyl-benzyl)-(pyridine-3-sulfonyl)-amino)-methyl)-phenoxy)-acetic acid sodium salt.

Form I, Method 1: Amorphous (3-(((4-tert-butyl-benzyl)-(pyridine-3-sulfonyl)-amino)-methyl)-phenoxy)-acetic acid sodium salt was prepared by first adding excess crystalline Form A to an organic solvent (acetonitrile or dichloromethane) and stirring at room temperature for 72 hours. The samples were then filtered and the solution was evaporated at room temperature under ambient conditions to yield a white solid, which was determined to be an amorphous solid by PXRD analysis.

Form I, Method 2: Amorphous (3-(((4-tert-butyl-benzyl)-(pyridine-3-sulfonyl)-amino)-methyl)-phenoxy)-acetic acid sodium salt was prepared by first adding excess crystalline Form A to one of the following solvent systems (Isoprypyl ether, Methyl t-butyl ether, or 97% ethyl acetate/3% water) and stirring at 40° C. for 72 hours. The samples were then filtered and the solution was evaporated at 40° C. to yield a white solid, which was determined to be an amorphous solid by PXRD analysis.

The polymorphic forms of (3-(((4-tert-butyl-benzyl)-(pyridine-3-sulfonyl)-amino)-methyl)-phenoxy)-acetic acid sodium salt described above are useful as prostaglandin agonists and therefore are useful in methods using such prostaglandin agonists and pharmaceutical compositions containing such prostaglandin agonists. The polymorphic forms of (3-(((4-tert-butyl-benzyl)-(pyridine-3-sulfonyl)-amino)-methyl)-phenoxy)-acetic acid sodium salt are useful in the treatment and/or prevention of conditions mediated by the modulation of prostaglandin, particularly conditions mediated by agonists of the EP₂ receptor.

It will be recognized that the polymorphic forms of (3-(((4-tert-butyl-benzyl)-(pyridine-3-sulfonyl)-amino)-methyl)-phenoxy)-acetic acid sodium salt of this invention can exist in radiolabeled form, i.e., said compounds may contain one or more atoms containing an atomic mass or mass number different from the atomic mass or mass number ordinarily found in nature. Radioisotopes of hydrogen, carbon, phosphorus, fluorine and chlorine include ³H, ¹⁴C, ³²P, ³⁵S, ¹⁸F and ³⁶Cl, respectively. Compounds of this invention which contain those radioisotopes and/or other radioisotopes of other atoms are within the scope of this invention. Tritiated, i.e., ³H, and carbon-14, i.e., ¹⁴C, radioisotopes are particularly preferred for their ease of preparation and detectability. Radiolabeled polymorphic forms of (3-(((4-tert-butyl-benzyl)-(pyridine-3-sulfonyl)-amino)-methyl)-phenoxy)-acetic acid sodium salt of this invention can generally be prepared of methods well known to those skilled in the art. Conveniently, such radiolabeled compounds can be prepared by carrying out the procedures disclosed in the above Schemes and/or in the Examples by substituting a readily available radiolabeled reagent for a non-radiolabeled reagent.

Those skilled in the art will recognize that anti-resorptive agents (for example progestins, polyphosphonates, bisphosphonate(s), estrogen agonists/antagonists, estrogen, estrogen/progestin combinations, Premarin®, estrone, estriol or 17α- or 17β-ethynyl estradiol) may be used in conjunction with the compounds of this invention.

Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol.

Preferred progestins are medroxyprogestrone, norethindrone and norethynodrel.

Exemplary bone resorption inhibiting polyphosphonates include polyphosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate is an especially preferred polyphosphonate. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl) amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

In particular, the compounds of this invention may be combined with a mammalian estrogen agonist/antagonist. Any estrogen agonist/antagonist may be used as the second compound of this invention. The term estrogen agonist/antagonist refers to compounds which bind with the estrogen receptor, inhibit bone turnover and/or prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods, and Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below.

A preferred estrogen agonist/antagonist is droloxifene: (phenol, 3-(1-(4-(2-(dimethylamino)ethoxy)phenyl)-2-phenyl-1-butenyl)-, (E)-) and related compounds which are disclosed in U.S. Pat. No. 5,047,431, the disclosure of which is incorporated herein by reference.

Another preferred estrogen agonist/antagonist is 3-(4-(1,2-diphenyl-but-1-enyl)-phenyl)-acrylic acid, which is disclosed in Willson et al., Endocrinology, 1997, 138, 3901-3911.

Another preferred estrogen agonist/antagonist is tamoxifen: (ethanamine, 2-(-4-(1,2-diphenyl-1-butenyl)phenoxy)-N,N-dimethyl, (Z)-2-, 2-hydroxy-1,2,3-propanetricarboxylate (1:1)) and related compounds which are disclosed in U.S. Pat. No. 4,536,516, the disclosure of which is incorporated herein by reference.

Another related compound is 4-hydroxy tamoxifen which is disclosed in U.S. Pat. No. 4,623,660, the disclosure of which is incorporated herein by reference.

A preferred estrogen agonist/antagonist is raloxifene: (methanone, (6-hydroxy-2-(4-hydroxyphenyl)benzo[b]thien-3-yl)(4-(2-(1-piperidinyl)ethoxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) which is disclosed in U.S. Pat. No. 4,996,225, the disclosure of which is incorporated herein by reference.

Another preferred estrogen agonist/antagonist is centchroman: 1-(2-((4-(-methoxy-2,2, dimethyl-3-phenyl-chroman-4-yl)-phenoxy)-ethyl)-pyrrolidine, which is disclosed in U.S. Pat. No. 3,822,287, the disclosure of which is incorporated herein by reference. Also preferred is levormeloxifene.

Another preferred estrogen agonist/antagonist is idoxifene: (E)-1-(2-(4-(1-(4-iodo-phenyl)-2-phenyl-but-1-enyl)-phenoxy)-ethyl)-pyrrolidinone, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thiophen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)-benzyl)-naphthalen-2-ol which is disclosed in U.S. Pat. No. 5,484,795, the disclosure of which is incorporated herein by reference.

Another preferred estrogen agonist/antagonist is (4-(2-(2-aza-bicyclo[2.2.1]hept-2-yl)-ethoxy)-phenyl)-(6-hydroxy-2-(4-hydroxy-phenyl)-benzo[b]thiophen-3-yl)-methanone which is disclosed, along with methods of preparation, in PCT publication no. WO 95/10513 assigned to Pfizer Inc.

Other preferred estrogen agonist/antagonists include compounds as described in commonly assigned U.S. Pat. No. 5,552,412, the disclosure of which is incorporated herein by reference. Especially preferred compounds described therein are:

cis-6-(4-fluoro-phenyl)-5-(4-(2-piperidin-1-yl-ethoxy)-phenyl)-5,6,7,8-tetrahydro-naphthalene-2-ol;

(−)-cis-6-phenyl-5-(4-(2-pyrrolidin-1-yl-ethoxy)-phenyl)-5,6,7,8-tetrahydro-naphthalene-2-ol;

cis-6-phenyl-5-(4-(2-pyrrolidin-1-yl-ethoxy)-phenyl)-5,6,7,8-tetrahydro-naphthalene-2-ol;

cis-1-(6′-pyrrolodinoethoxy-3′-pyridyl)-2-phenyl-6-hydroxy-1,2,3,4-tetrahydronaphthalene;

1-(4′-pyrrolidinoethoxyphenyl)-2-(4″-fluorophenyl)-6-hydroxy-1,2,3,4-tetrahydroisoquinoline;

cis-6-(4-hydroxyphenyl)-5-(4-(2-piperidin-1-yl-ethoxy)-phenyl)-5,6,7,8-tetrahydro-naphthalene-2-ol; and

1-(4′-pyrrolidinolethoxyphenyl)-2-phenyl-6-hydroxy-1,2,3,4-tetrahydroisoquinoline.

Other estrogen agonist/antagonists are described in U.S. Pat. No. 4,133,814 (the disclosure of which is incorporated herein by reference). U.S. Pat. No. 4,133,814 discloses derivatives of 2-phenyl-3-aroyl-benzothiophene and 2-phenyl-3-aroylbenzothiophene-1-oxide.

Those skilled in the art will recognize that other bone anabolic agents, also referred to as bone mass augmenting agents, may be used in conjunction with the compounds of this invention. A bone mass augmenting agent is a compound that augments bone mass to a level which is above the bone fracture threshold as detailed in the World Health Organization Study World Health Organization, “Assessment of Fracture Risk and its Application to Screening for Postmenopausal Osteoporosis (1994). Report of a WHO Study Group. World Health Organization Technical Series 843.”

Any prostaglandin, or prostaglandin agonist/antagonist may be used as the second compound in certain aspects of this invention. Those skilled in the art will recognize that IGF-1, sodium fluoride, parathyroid hormone (PTH), active fragments of parathyroid hormone, growth hormone or growth hormone secretagogues may also be used. The following paragraphs describe exemplary second compounds of this invention in greater detail.

Any prostaglandin may be used as the second compound in certain aspects of this invention. The term prostaglandin refers to compounds which are analogs of the natural prostaglandins PGD₁, PGD₂, PGE₂, PGE₁ and PGF₂ which are useful in the treatment of osteoporosis. These compounds bind to the prostaglandin receptors. Such binding is readily determined by those skilled in the art of standard assays (e.g., An S. et al., Cloning and Expression of the EP₂ Subtype of Human Receptors for Prostaglandin E₂, Biochemical and Biophysical Research Communications, 1993, 197(1):263-270).

Prostaglandins are alicyclic compounds related to the basic compound prostanoic acid. The carbon atoms of the basic prostaglandin are numbered sequentially from the carboxylic carbon atom through the cyclopentyl ring to the terminal carbon atom on the adjacent side chain. Normally the adjacent side chains are in the trans orientation. The presence of an oxo group at C-9 of the cyclopentyl moiety is indicative of a prostaglandin within the E class while PGE₂ contains a trans unsaturated double bond at the C₁₃-C₁₄ and a cis double bond at the C₅-C₆ position.

A variety of prostaglandins are described and referenced below. However, other prostaglandins will be known to those skilled in the art. Exemplary prostaglandins are disclosed in U.S. Pat. Nos. 4,171,331 and 3,927,197, the disclosures of each of which are incorporated herein by reference.

Norrdin et al., The Role of Prostaglandins in Bone In Vivo, Prostaglandins Leukotriene Essential Fatty Acids 41, 139-150, 1990 is a review of bone anabolic prostaglandins.

Any prostaglandin agonist/antagonist may be used as the second compound in certain aspects of this invention. The term prostaglandin agonist/antagonist refers to compounds which bind to prostaglandin receptors (e.g., An S. et al., Cloning and Expression of the EP₂ Subtype of Human Receptors for Prostaglandin E₂, Biochemical and Biophysical Research Communications, 1993, 197(1):263-270) and mimic the action of prostaglandin in vivo (e.g., stimulate bone formation and increase bone mass). Such actions are readily determined by those skilled in the art of standard assays. Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296. A variety of these compounds are described and referenced below. However, other prostaglandin agonists/antagonists will be known to those skilled in the art. Exemplary prostaglandin agonists/antagonists are disclosed as follows.

Commonly assigned U.S. Pat. No. 3,932,389, the disclosure of which is incorporated herein by reference, discloses 2-descarboxy-2-(tetrazol-5-yl)-11-desoxy-15-substituted-omega-pentanorprostaglandins useful for bone formation activity.

Commonly assigned U.S. Pat. No. 4,018,892, the disclosure of which is incorporated herein by reference, discloses 16-aryl-13,14-dihydro-PGE₂ p-biphenyl esters useful for bone formation activity.

Commonly assigned U.S. Pat. No. 4,219,483, the disclosure of which is incorporated herein by reference, discloses 2,3,6-substituted-4-pyrones useful for bone formation activity.

Commonly assigned U.S. Pat. No. 4,132,847, the disclosure of which is incorporated herein by reference, discloses 2,3,6-substituted-4-pyrones useful for bone formation activity.

U.S. Pat. No. 4,000,309, the disclosure of which is incorporated herein by reference, discloses 16-aryl-13,14-dihydro-PGE₂ p-biphenyl esters useful for bone formation activity.

U.S. Pat. No. 3,982,016, the disclosure of which is incorporated herein by reference, discloses 16-aryl-13,14-dihydro-PGE₂ p-biphenyl esters useful for bone formation activity.

U.S. Pat. No. 4,621,100, the disclosure of which is incorporated herein by reference, discloses substituted cyclopentanes useful for bone formation activity.

U.S. Pat. No. 5,216,183, the disclosure of which is incorporated herein by reference, discloses cyclopentanones useful for bone formation activity.

Sodium fluoride may be used as the second compound in certain aspects of this invention. The term sodium fluoride refers to sodium fluoride in all its forms (e.g., slow release sodium fluoride, sustained release sodium fluoride). Sustained release sodium fluoride is disclosed in U.S. Pat. No. 4,904,478, the disclosure of which is incorporated herein by reference. The activity of sodium fluoride is readily determined by those skilled in the art of biological protocols (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296).

Bone morphogenetic protein may be used as the second compound of this invention (e.g., see Ono, et al., Promotion of the Osteogenetic Activity of Recombinant Human Bone Morphogenetic Protein by Prostaglandin E1, Bone, 1996, 19(6), 581-588).

Any parathyroid hormone (PTH) may be used as the second compound in certain aspects of this invention. The term parathyroid hormone refers to parathyroid hormone, fragments or metabolites thereof and structural analogs thereof which can stimulate bone formation and increase bone mass. Also included are parathyroid hormone related peptides and active fragments and analogs of parathyroid related peptides (see PCT publication no. WO 94/01460). Such bone anabolic functional activity is readily determined by those skilled in the art of standard assays (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below. However, other parathyroid hormones will be known to those skilled in the art. Exemplary parathyroid hormones are disclosed in the following references.

“Human Parathyroid Peptide Treatment of Vertebral Osteoporosis”, Osteoporosis Int., 3, (Supp 1):199-203.

“PTH 1-34 Treatment of Osteoporosis with Added Hormone Replacement Therapy: Biochemical, Kinetic and Histological Responses” Osteoporosis Int. 1:162-170.

Any growth hormone or growth hormone secretagogue may be used as the second compound in certain aspects of this invention. The term growth hormone secretagogue refers to a compound which stimulates the release of growth hormone or mimics the action of growth hormone (e.g., increases bone formation leading to increased bone mass). Such actions are readily determined by those skilled in the art of standard assays well known to those of skill in the art. A variety of these compounds are disclosed in the following published PCT patent applications: WO 95/14666; WO 95/13069; WO 94/19367; WO 94/13696; and WO 95/34311. However, other growth hormones or growth hormone secretagogues will be known to those skilled in the art.

In particular a preferred growth hormone secretagogue is N-[1(R)-[1,2-Dihydro-1-methanesulfonylspiro[3H-indole-3,4′-piperidin]-1′-yl)carbonyl]-2-(phenylmethyloxy)ethyl]-2-amino-2-methylpropanamide: MK-677.

Other preferred growth hormone secretagogues include

2-amino-N-(2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl)-isobutyramide or its L-tartaric acid salt;

2-amino-N-(1-(R)-benzyloxymethyl-2-(3a-(R)-(4-fluoro-benzyl)-2-methyl-3-oxo-2,3,3a,4,6,7-hexahydro-pyrazolo[4,3-c]pyridin-5-yl)-2-oxo-ethyl)isobutyramide;

2-amino-N-(2-(3a-(R)-benzyl-3-oxo-2,3,3a,4,6,7-hexahydro-pyrazolo[4,3-c]pyridin-5-yl)-1-(R)benzyloxymethyl-2-oxo-ethyl)isobutyramide; and

2-amino-N-(1-(2,4-difluoro-benzyloxymethyl)-2-oxo-2-(3-oxo-3a-pyridin-2-ylmethyl-2-(2,2,2-trifluoro-ethyl)-2,3,3a,4,6,7-hexahydro-pyrazolo[4,3-c]pyridin-5-yl)-ethyl)-2-methyl-propionamide.

Some of the preparation methods useful for the preparation of the compounds described herein may require protection of remote functionality (e.g., primary amine, secondary amine, carboxyl in Formula I precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

The compounds of this invention, prodrugs thereof and pharmaceutically acceptable salts of said compounds and prodrugs are all adapted to therapeutic use as agents that stimulate bone formation and increase bone mass in a vertebrates, e.g., mammals, and particularly humans. Since bone formation is closely related to the development of osteoporosis and bone related disorders, these compounds, prodrugs thereof and pharmaceutically acceptable salts of said compounds and said prodrugs, by virtue of their action on bone, prevent, arrest and/or regress osteoporosis.

The utility of the compounds of the present invention as medical agents in the treatment of conditions which present with low bone mass (e.g., osteoporosis) in a vertebrates, e.g., mammals (e.g. humans, particularly the female) is demonstrated by the activity of the compounds of this invention in conventional assays, including an in vivo assay, a receptor binding assay, a cyclic AMP assay and a fracture healing assay (all of which are described below). The in vivo assay (with appropriate modifications within the skill in the art) may be used to determine the activity of other anabolic agents as well as the prostaglandin agonists of this invention. The estrogen agonist/antagonist protocol may be used to determine the activity of estrogen agonists/antagonists in particular and also other anti-resorptive agents (with appropriate modifications within the skill in the art). The combination and sequential treatment protocol described below is useful for demonstrating the utility of the combinations of the anabolic agents (e.g., the compounds of this invention) and anti-resorptive agents (e.g., estrogen agonists/antagonists) described herein. Such assays also provide a means whereby the activities of the compounds of this invention (or the other anabolic agents and anti-resorptive agents described herein) can be compared to each other and with the activities of other known compounds. The results of these comparisons are useful for determining dosage levels in a vertebrates, e.g., mammals, including humans, for the treatment of such diseases.

### Anabolic Agent in Vivo Assay

The activity of anabolic bone agents in stimulating bone formation and increasing bone mass can be tested in intact male or female rats, sex hormone deficient male (orchidectomy) or female (ovariectomy) rats.

Male or female rats at different ages (such as 3 months of age) can be used in the study. The rats are either intact or castrated (ovariectomized or orchidectomized), and subcutaneously injected or gavaged with prostaglandin agonists at different doses (such as 1, 3, or 10 mg/kg/day) for 30 days. In the castrated rats, treatment is started at the next day after surgery (for the purpose of preventing bone loss) or at the time bone loss has already occurred (for the purpose of restoring bone mass). During the study, all rats are allowed free access to water and a pelleted commercial diet (Teklad Rodent Diet #8064, Harlan Teklad, Madison, Wis.) containing 1.46% calcium, 0.99% phosphorus and 4.96 IU/g of Vitamin D₃. All rats are given subcutaneous injections of 10 mg/kg calcein on days 12 and 2 before sacrifice. The rats are sacrificed. The following endpoints are determined:

### Femoral Bone Mineral Measurements:

The right femur from each rat is removed at autopsy and scanned using dual energy X-ray absorptiometry (DXA, QDR 1000/W, Hologic Inc., Waltham, Mass.) equipped with “Regional High Resolution Scan” software (Hologic Inc., Waltham, Mass.). The scan field size is 5.08×1.902 cm, resolution is 0.0254×0.0127 cm and scan speed is 7.25 mm/second. The femoral scan images are analyzed and bone area, bone mineral content (BMC), and bone mineral density (BMD) of whole femora (WF), distal femoral metaphyses (DFM), femoral shaft (FS), and proximal femora (PF) are determined.

### Tibial Bone Histomorphometric Analyses:

The right tibia is removed at autopsy, dissected free of muscle, and cut into three parts. The proximal tibia and the tibial shaft are fixed in 70% ethanol, dehydrated in graded concentrations of ethanol, defatted in acetone, then embedded in methyl methacrylate (Eastman Organic Chemicals, Rochester, N.Y.).

Frontal sections of proximal tibial metaphyses at 4 and 10 μm thickness are cut using a Reichert-Jung Polycut S microtome. The 4 μm sections are stained with modified Masson's Trichrome stain while the 10 μm sections remained unstained. One 4 μm and one 10 μm sections from each rat are used for cancellous bone histomorphometry.

Cross sections of tibial shaft at 10 μm thickness are cut using a Reichert-Jung Polycut S microtome. These sections are used for cortical bone histomorphometric analysis.

Cancellous bone histomorphometry: A Bioquant OS/2 histomorphometry system (R&M Biometrics, Inc., Nashville, Tenn.) is used for the static and dynamic histomorphometric measurements of the secondary spongiosa of the proximal tibial metaphyses between 1.2 and 3.6 mm distal to the growth plate-epiphyseal junction. The first 1.2 mm of the tibial metaphyseal region needs to be omitted in order to restrict measurements to the secondary spongiosa. The 4 μm sections are used to determine indices related to bone volume, bone structure, and bone resorption, while the 10 μm sections are used to determine indices related to bone formation and bone turnover.

I) Measurements and calculations related to trabecular bone volume and structure: (1) Total metaphyseal area (TV, mm²): metaphyseal area between 1.2 and 3.6 mm distal to the growth plate-epiphyseal junction. (2) Trabecular bone area (BV, mm²): total area of trabeculae within TV. (3) Trabecular bone perimeter (BS, mm): the length of total perimeter of trabeculae. (4) Trabecular bone volume (BV/TV, %): BV/TV×100. (5) Trabecular bone number (TBN, #/mm): 1.199/2×BS/TV. (6) Trabecular bone thickness (TBT, μm): (2000/1.199)×(BV/BS). (7) Trabecular bone separation (TBS, μm): (2000×1.199)×(TV−BV).

II) Measurements and calculations related to bone resorption: (1) Osteoclast number (OCN, #): total number of osteoclast within total metaphyseal area. (2) Osteoclast perimeter (OCP, mm): length of trabecular perimeter covered by osteoclast. (3) Osteoclast number/mm (OCN/mm, #/mm): OCN/BS. (4) Percent osteoclast perimeter (% OCP, %): OCP/BS×100.

III) Measurements and calculations related to bone formation and turnover: (1) Single-calcein labeled perimeter (SLS, mm): total length of trabecular perimeter labeled with one calcein label. (2) Double-calcein labeled perimeter (DLS, mm): total length of trabecular perimeter labeled with two calcein labels. (3) Inter-labeled width (ILW, μm): average distance between two calcein labels. (4) Percent mineralizing perimeter (PMS, %): (SLS/2+DLS)/BS×100. (5) Mineral apposition rate (MAR, μm/day): ILW/label interval. (6) Bone formation rate/surface ref. (BFR/BS, μm²/d/μm): (SLS/2+DLS)×MAR/BS. (7) Bone turnover rate (BTR, %/y): (SLS/2+DLS)×MAR/BV×100.

Cortical bone histomorphometry: A Bioquant OS/2 histomorphometry system (R&M Biometrics, Inc., Nashville, Tenn.) is used for the static and dynamic histomorphometric measurements of tibial shaft cortical bone. Total tissue area, marrow cavity area, periosteal perimeter, endocortical perimeter, single labeled perimeter, double labeled perimeter, and interlabeled width on both periosteal and endocortical surface are measured, and cortical bone area (total tissue area−marrow cavity area), percent cortical bone area (cortical area/total tissue area×100), percent marrow area (marrow cavity area/total tissue area×100), periosteal and endocortical percent labeled perimeter [(single labeled perimeter/2+double labeled perimeter)/total perimeter×100], mineral apposition rate (interlabeled width/intervals), and bone formation rate [mineral apposition rate×[(single labeled perimeter/2+double labeled perimeter)/total perimeter] are calculated.

### Statistics

Statistics can be calculated using StatView 4.0 packages (Abacus Concepts, Inc., Berkeley, Calif.). The analysis of variance (ANOVA) test followed by Fisher's PLSD (Stat View, Abacus Concepts Inc., 1918 Bonita Ave, Berkeley, Calif. 94704-1014) are used to compare the differences between groups.

### Determination of cAMP Elevation in 293-S Cell Lines Stably Overexpressing Recombinant Human EP₂ and EP₄ Receptors

cDNAs representing the complete open reading frames of the human EP₂ and EP₄ receptors are generated by reverse transcriptase polymerase chain reaction using oligonucleotide primers based on published sequences (Regan, J. W. Bailey, T. J. Pepperl, D. J. Pierce, K. L. Bogardus, A. M. Donello, J. E. Fairbairn, C. E. Kedzie, K. M. Woodward, D. F. and Gil, D. W. 1994 Cloning of a Novel Human Prostaglandin Receptor with Characteristics of the Pharmacologically Defined EP₂ Subtype. Mol. Pharmacology 46:213-220; and Bastien, L., Sawyer, N., Grygorczyk, R., Metters, K., and Adam, M. 1994 Cloning, Functional Expression, and Characterization of the Human Prostaglandin E2 Receptor EP2 Subtype. J. Biol. Chem. Vol 269, 16:11873-11877) and RNA from primary human kidney cells (EP₂) or primary human lung cells (EP₄) as templates. cDNAs are cloned into the multiple cloning site of pcDNA3 (Invitrogen Corporation, 3985B Sorrento Valley Blvd., San Diego, Calif. 92121) and used to transfect 293-S human embryonic kidney cells via calcium phosphate co-precipitation. G418-resistant colonies are expanded and tested for specific [³H]PGE₂ binding. Transfectants demonstrating high levels of specific [³H]PGE₂ binding are further characterized by Scatchard analysis to determine Bmax and Kds for PGE₂. The lines selected for compound screening have approximately 338,400 receptors per cell and a Kd=12 nM for PGE₂ (EP₂), and approximately 256,400 receptors per cell and a Kd=2.9 nM for PGE₂ (EP₄). Constituitive expression of both receptors in parental 293-S cells is negligible. Cells are maintained in RPMI supplemented with fetal bovine serum (10% final) and G418 (700 ug/ml final).

cAMP responses in the 293-S/EP₂ and 293-S/EP₄ lines are determined by detaching cells from culture flasks in 1 ml of Ca++ and Mg++ deficient PBS via vigorous pounding, adding serum-free RPMI to a final concentration of 1×10⁶ cells/ml, and adding 3-isobutyl-1-methylxanthine (IBMX) to a final concentration of 1 mM. One milliliter of cell suspension is immediately aliquoted into individual 2 ml screwcap microcentrifuge and incubated for 10 minutes, uncovered, at 37° C., 5% CO₂, 95% relative humdity. The compound to be tested is then added to cells at 1:100 dilutions such that final DMSO or ethanol concentrations is 1%. Immediately after adding compound, the tubes are covered, mixed by inverting two times, and incubated at 37° C. for 12 minutes. Samples are then lysed by incubation at 100° C. for 10 minutes and immediately cooled on ice for 5 minutes. Cellular debris is pelleted by centrifugation at 1000×g for 5 minutes, and cleared lysates are transferred to fresh tubes. cAMP concentrations are determined using a commercially available cAMP radioimmunoassay kit RIA (NEK-033, DuPont/NEN Research Products, 549 Albany St., Boston, Mass. 02118) after diluting cleared lysates 1:10 in cAMP RIA assay buffer (included in kit). Typically, one treats cells with 6-8 concentrations of the compound to be tested in 1 log increments. EC50 calculations are performed on a calculator using linear regression analysis on the linear portion of the dose response curves.

### Assay for Binding to Prostaglandin E₂ Receptors

Membrane Preparation: All operations are performed at 4° C. Transfected cells expressing prostaglandin E₂ type 1 receptors (EPA type 2 (EP₂), type 3 (EP₃) or type 4 (EP₄) receptors are harvested and suspended to 2 million cells per ml in Buffer A [50 mM Tris-HCl (pH 7.4), 10 mM MgCl₂, 1 mM EDTA, 1 mM Pefabloc peptide, (Boehringer Mannheim Corp., Indianapolis, Ind.), 10 uM Phosporamidon peptide, (Sigma, St. Louis, Mo.), 1 uM pepstatin A peptide, (Sigma, St. Louis, Mo.), 10 uM elastatinal peptide, (Sigma, St. Louis, Mo.), 100 uM antipain peptide, (Sigma, St. Louis, Mo.)]. The cells are lysed by sonification with a Branson Sonifier (Model #250, Branson Ultrasonics Corporation, Danbury, Conn.) in 2 fifteen second bursts. Unlysed cells and debris are removed by centrifugation at 100×g for 10 min. Membranes are then harvested by centrifugation at 45,000×g for 30 minutes. Pelleted membranes are resuspended to 3-10 mg protein per ml, protein concentration being determined of the method of Bradford [Bradford, M., Anal. Biochem., 72, 248 (1976)]. Resuspended membranes are then stored frozen at −80° C. until use.

Binding Assay: Frozen membranes prepared as above are thawed and diluted to 1 mg protein per ml in Buffer A above. One volume of membrane preparation is combined with 0.05 volume test compound or buffer and one volume of 3 nM ³H-prostaglandin E₂ (#TRK 431, Amersham, Arlington Heights, Ill.) in Buffer A. The mixture (205 μL total volume) is incubated for 1 hour at 25° C. The membranes are then recovered by filtration through type GF/C glass fiber filters (#1205-401, Wallac, Gaithersburg, Md.) using a Tomtec harvester (Model Mach II/96, Tomtec, Orange, Conn.). The membranes with bound ³H-prostaglandin E₂ are trapped by the filter, while the buffer and unbound ³H-prostaglandin E₂ pass through the filter into waste. Each sample is then washed 3 times with 3 ml of [50 mM Tris-HCl (pH 7.4), 10 mM MgCl₂, 1 mM EDTA]. The filters are then dried by heating in a microwave oven. To determine the amount of ³H-prostaglandin bound to the membranes, the dried filters are placed into plastic bags with scintillation fluid and counted in a LKB 1205 Betaplate reader (Wallac, Gaithersburg, Md.). IC50s are determined from the concentration of test compound required to displace 50% of the specifically bound ³H-prostaglandin E₂.

The full length EP₁ receptor is made as disclosed in Funk et al., Journal of Biological Chemistry, 1993, 268, 26767-26772. The full length EP₂ receptor is made as disclosed in Regan et al., Molecular Pharmacology, 1994, 46, 213-220. The full length EP₃ receptor is made as disclosed in Regan et al., British Journal of Pharmacology, 1994, 112, 377-385. The full length EP₄ receptor is made as disclosed in Bastien, Journal of Biological Chemistry, 1994, 269, 11873-11877. These full length receptors are used to prepare 293S cells expressing the EP₁, EP₂, EP₃ and EP₄ receptors.

293S cells expressing either the human EP₁, EP₂, EP₃ or EP₄ prostaglandin E₂ receptors are generated according to methods known to those skilled in the art. Typically, PCR (polymerase chain reaction) primers corresponding to the 5′ and 3′ ends of the published full length receptor are made according to the well known methods disclosed above and are used in an RT-PCR reaction using the total RNA from human kidney (for EPA human lung (for EP₂), human lung (for EP₃) or human lymphocytes (for EP₄) as a source. PCR products are cloned by the TA overhang method into pCR2.1 (Invitrogen, Carlsbad, Calif.) and identity of the cloned receptor is confirmed by DNA sequencing.

293S cells (Mayo, Dept. of Biochemistry, Northwestern Univ.) are transfected with the cloned receptor in pcDNA3 by electroporation. Stable cell lines expressing the receptor are established following selection of transfected cells with G418.

Clonal cell lines expressing the maximal number of receptors are chosen following a whole cell ³H-PGE₂ binding assay using unlabeled PGE₂ as a competitor.

### Fracture Healing Assays

### Assay for Effects on Fracture Healing After Systemic Administration

Fracture Technique: Sprage-Dawley rats at 3 months of age are anesthetized with Ketamine. A 1 cm incision is made on the anteromedial aspect of the proximal part of the right tibia or femur. The following describes the tibial surgical technique. The incision is carried through to the bone, and a 1 mm hole is drilled 4 mm proximal to the distal aspect of the tibial tuberosity 2 mm medial to the anterior ridge. Intramedullary nailing is performed with a 0.8 mm stainless steel tube (maximum load 36.3 N, maximum stiffness 61.8 N/mm, tested under the same conditions as the bones). No reaming of the medullary canal is performed. A standardized closed fracture is produced 2 mm above the tibiofibular junction by three-point bending using specially designed adjustable forceps with blunt jaws. To minimize soft tissue damage, care is taken not to displace the fracture. The skin is closed with monofilament nylon sutures. The operation is performed under sterile conditions. Radiographs of all fractures are taken immediately after nailing, and rats with fractures outside the specified diaphyseal area or with displaced nails are excluded. The remaining animals are divided randomly into the following groups with 10-12 animals per each subgroup per time point for testing the fracture healing. The first group receives daily gavage of vehicle (water:100% Ethnanol=95:5) at 1 ml/rat, while the others receive daily gavage from 0.01 to 100 mg/kg/day of the compound to be tested (1 ml/rat) for 10, 20, 40 and 80 days.

At 10, 20, 40 and 80 days, 10-12 rats from each group are anesthetized with Ketamine and sacrificed by exsanguination. Both tibiofibular bones are removed by dissection and all soft tissue is stripped. Bones from 5-6 rats for each group are stored in 70% ethanol for histological analysis, and bones from another 5-6 rats for each group are stored in a buffered Ringer's solution (+4° C., pH 7.4) for radiographs and biomechanical testing which is performed.

Histological Analysis: The methods for histologic analysis of fractured bone have been previously published by Mosekilde and Bak (The Effects of Growth Hormone on Fracture Healing in Rats: A Histological Description. Bone, 14:19-27, 1993). Briefly, the fracture side is sawed 8 mm to each side of the fracture line, embedded undecalcified in methymethacrylate, and cut frontals sections on a Reichert-Jung Polycut microtome in 8 μm thick. Masson-Trichrome stained mid-frontal sections (including both tibia and fibula) are used for visualization of the cellular and tissue response to fracture healing with and without treatment. Sirius red stained sections are used to demonstrate the characteristics of the callus structure and to differentiate between woven bone and lamellar bone at the fracture site. The following measurements are performed: (1) fracture gap—measured as the shortest distance between the cortical bone ends in the fracture, (2) callus length and callus diameter, (3) total bone volume area of callus, (4) bony tissue per tissue area inside the callus area, (5) fibrous tissue in the callus, and (6) cartilage area in the callus.

Biomechanical Analysis: The methods for biomechanical analysis have been previously published by Bak and Andreassen (The Effects of Aging on Fracture Healing in Rats. Calcif Tissue Int 45:292-297, 1989). Briefly, radiographs of all fractures are taken prior to the biomechanical test. The mechanical properties of the healing fractures are analyzed by a destructive three- or four-point bending procedure. Maximum load, stiffness, energy at maximum load, deflection at maximum load, and maximum stress are determined.

### Assay for Effects on Fracture Healing After Local Administration

Fracture Technique: Female or male beagle dogs at approximately 2 years of age are used under anesthesia in the study. Transverse radial fractures are produced by slow continuous loading in three-point bending as described by Lenehan et al. (Lenehan, T. M.; Balligand, M.; Nunamaker, D. M.; Wood, F. E.: Effects of EHDP on Fracture Healing in Dogs. J Orthop Res 3:499-507; 1985). The wire is pulled through the fracture site to ensure complete anatomical disruption of the bone. Thereafter, local delivery of prostaglandin agonists to the fracture site is achieved by slow release of compound delivered by slow release pellets or by administration of the compounds in a suitable Formulation such as a paste gel solution or suspension for 10, 15, or 20 weeks.

Histological Analysis: The methods for histologic analysis of fractured bone have been previously published by Peter et al. (Peter, C. P.; Cook, W. O.; Nunamaker, D. M.; Provost, M. T.; Seedor, J. G.; Rodan, G. A. Effects of alendronate on fracture healing and bone remodeling in dogs. J. Orthop. Res. 14:74-70, 1996) and Mosekilde and Bak (The Effects of Growth Hormone on Fracture Healing in Rats: A Histological Description. Bone, 14:19-27, 1993). Briefly, after sacrifice, the fracture side is sawed 3 cm to each side of the fracture line, embedded undecalcified in methymethacrylate, and cut on a Reichert-Jung Polycut microtome in 8 μm thick of frontal sections. Masson-Trichrome stained mid-frontal sections (including both tibia and fibula) are used for visualization of the cellular and tissue response to fracture healing with and without treatment. Sirius red stained sections are used to demonstrate the characteristics of the callus structure and to differentiate between woven bone and lamellar bone at the fracture site. The following measurements are performed: (1) fracture gap—measured as the shortest distance between the cortical bone ends in the fracture, (2) callus length and callus diameter, (3) total bone volume area of callus, (4) bony tissue per tissue area inside the callus area, (5) fibrous tissue in the callus, (6) cartilage area in the callus.

Biomechanical Analysis: The methods for biomechanical analysis have been previously published by Bak and Andreassen (The Effects of Aging on Fracture Healing in Rats. Calcif Tissue Int 45:292-297, 1989) and Peter et al. (Peter, C. P.; Cook, W. O.; Nunamaker, D. M.; Provost, M. T.; Seedor, J. G.; Rodan, G. A. Effects of Alendronate On Fracture Healing And Bone Remodeling In Dogs. J. Orthop. Res. 14:74-70, 1996). Briefly, radiographs of all fractures are taken prior to the biomechanical test. The mechanical properties of the healing fractures are analyzed by a destructive three- or four-point bending procedures. Maximum load, stiffness, energy at maximum load, deflection at maximum load, and maximum stress are determined.

### Estrogen Agonist/Antagonist Protocol

Estrogen agonist/antagonists are a class of compounds which inhibit bone turnover and prevent estrogen-deficiency induced bone loss. The ovariectomized rat bone loss model has been widely used as a model of postmenopausal bone loss. Using this model, one can test the efficacy of the estrogen agonist/antagonist compounds in preventing bone loss and inhibiting bone resorption.

Sprague-Dawley female rats (Charles River, Wilmington, Mass.) at different ages (such as 5 months of age) are used in these studies. The rats are singly housed in 20 cm×32 cm×20 cm cages during the experimental period. All rats are allowed free access to water and a pelleted commercial diet (Agway ProLab 3000, Agway County Food, Inc., Syracuse, N.Y.) containing 0.97% calcium, 0.85% phosphorus, and 1.05 IU/g of Vitamin D₃

A group of rats (8 to 10) are sham-operated and treated p.o. with vehicle (10% ethanol and 90% saline, 1 ml/day), while the remaining rats are bilaterally ovariectomized (OVX) and treated with either vehicle (p.o.), 17β-estradiol (Sigma, E-8876, E₂, 30 μg/kg, daily subcutaneous injection), or estrogen agonist/antagonists (such as droloxifene at 5, 10, or 20 mg/kg, daily p.o.) for a certain period (such as 4 weeks). All rats are given subcutaneous injections of 10 mg/kg calcein (fluorochrome bone marker) 12 and 2 days before being sacrificed in order to examine the dynamic changes in bone tissue. After 4 weeks of treatment, the rats are sacrificed and autopsied. The following endpoints are determined:

Body Weight Gain: Body weight at autopsy minus body weight at surgery.

Uterine Weight and Histology: The uterus is removed from each rat during autopsy, and weighed immediately. Thereafter, the uterus is processed for histologic measurements such as uterine cross-sectional tissue area, stromal thickness, and luminal epithelial thickness.

Total Serum Cholesterol: Blood is obtained by cardiac puncture and allowed to clot at 4° C., and then centrifuged at 2,000 g for 10 min. Serum samples are analyzed for total serum cholesterol using a high performance cholesterol calorimetric assay (Boehringer Mannheim Biochemicals, Indianapolis, Ind.).

Femoral Bone Mineral Measurements: The right femur from each rat is removed at autopsy and scanned using dual energy X-ray absorptiometry (DEXA, QDR 1000/W, Hologic Inc., Waltham, Mass.) equipped with “Regional High Resolution Scan” software (Hologic Inc., Waltham, Mass.). The scan field size is 5.08×1.902 cm, resolution is 0.0254×0.0127 cm and scan speed is 7.25 mm/second. The femoral scan images are analyzed and bone area, bone mineral content (BMC), and bone mineral density (BMD) of whole femora (WF), distal femoral metaphyses (DFM), femoral shaft (FS), and proximal femora (PF) are determined.

Proximal Tibial Metaphyseal Cancellous Bone Histomorphometric Analyses: The right tibia is removed at autopsy, dissected free of muscle, and cut into three parts. The proximal tibia is fixed in 70% ethanol, dehydrated in graded concentrations of ethanol, defatted in acetone, then embedded in methyl methacrylate (Eastman Organic Chemicals, Rochester, N.Y.). Frontal sections of proximal tibial metaphyses at 4 and 10 μm thickness are cut using a Reichert-Jung Polycut S microtome. One 4 μm and one 10 μm sections from each rat are used for cancellous bone histomorphometry. The 4 μm sections are stained with modified Masson's Trichrome stain while the 10 μm sections remained unstained.

A Bioquant OS/2 histomorphometry system (R&M Biometrics, Inc., Nashville, Tenn.) is used for the static and dynamic histomorphometric measurements of the secondary spongiosa of the proximal tibial metaphyses between 1.2 and 3.6 mm distal to the growth plate-epiphyseal junction. The first 1.2 mm of the tibial metaphyseal region is omitted in order to restrict measurements to the secondary spongiosa. The 4 μm sections are used to determine indices related to bone volume, bone structure, and bone resorption, while the 10 μm sections are used to determine indices related to bone formation and bone turnover.

### I. Measurements and Calculations Related to Trabecular Bone Volume and Structure:

1. Total metaphyseal area (TV, mm²): metaphyseal area between 1.2 and 3.6 mm distal to the growth plate-epiphyseal junction.

2. Trabecular bone area (BV, mm²): total area of trabeculae within TV.

3. Trabecular bone perimeter (BS, mm): the length of total perimeter of trabeculae.

4. Trabecular bone volume (BV/TV, %): BV/TV×100.

5. Trabecular bone number (TBN, #/mm): 1.199/2×BS/TV.

6. Trabecular bone thickness (TBT, μm): (2000/1.199)×(BV/BS).

7. Trabecular bone separation (TBS, μm): (2000×1.199)×(TV−BV).

### II. Measurements and Calculations Related to Bone Resorption:

1. Osteoclast number (OCN, #): total number of osteoclast within total metaphyseal area.

2. Osteoclast perimeter (OCP, mm): length of trabecular perimeter covered by osteoclast.

3. Osteoclast number/mm (OCN/mm, #/mm): OCN/BS.

4. Percent osteoclast perimeter (% OCP, %): OCP/BS×100.

### III. Measurements and Calculations Related to Bone Formation and Turnover:

1. Single-calcein labeled perimeter (SLS, mm): total length of trabecular perimeter labeled with one calcein label.

2. Double-calcein labeled perimeter (DLS, mm): total length of trabecular perimeter labeled with two calcein labels.

3. Inter-labeled width (ILW, μm): average distance between two calcein labels.

4. Percent mineralizing perimeter (PMS, %): (SLS/2+DLS)/BS×100.

5. Mineral apposition rate (MAR, μm/day): ILW/label interval.

6. Bone formation rate/surface ref. (BFR/BS, μm²/d/μm): (SLS/2+DLS)×MAR/BS.

7. Bone turnover rate (BTR, %/y): (SLS/2+DLS)×MAR/BV×100.

### Statistics

Statistics are calculated using StatView 4.0 packages (Abacus Concepts, Inc., Berkeley, Calif.). The analysis of variance (ANOVA) test followed by Fisher's PLSD (Stat View, Abacus Concepts Inc. 1918 Bonita Ave, Berkeley, Calif. 94704-1014) is used to compare the differences between groups.

### Combination and Sequential Treatment Protocol

The following protocols can of course be varied by those skilled in the art. For example, intact male or female rats, sex hormone deficient male (orchidectomy) or female (ovariectomy) rats may be used. In addition, male or female rats at different ages (such as 12 months of age) can be used in the studies. The rats can be either intact or castrated (ovariectomized or orchidectomized), and administered to with anabolic agents such as the compounds of this invention at different doses (such as 1, 3 or 6 mg/kg/day) for a certain period (such as two weeks to two months), and followed by administration of an anti-resorptive agent such as droloxifene at different doses (such as 1, 5, 10 mg/kg/day) for a certain period (such as two weeks to two months), or a combination treatment with both anabolic agent and anti-resorptive agent at different doses for a certain period (such as two weeks to two months). In the castrated rats, treatment can be started on the next day after surgery (for the purpose of preventing bone loss) or at the time bone loss has already occurred (for the purpose of restoring bone mass).

The rats are sacrificed under ketamine anesthesia. The following endpoints are determined:

Femoral bone mineral measurements are performed as described above in the estrogen agonist/antagonist protocol.

Lumbar Vertebral Bone Mineral Measurements: Dual energy X-ray absorptiometry (QDR 1000/W, Hologic, Inc., Waltham, Mass.) equipped with a “Regional High Resolution Scan” software (Hologic, Inc., Waltham, Mass.) is used to determined the bone area, bone mineral content (BMC), and bone mineral density (BMD) of whole lumbar spine and each of the six lumbar vertebrae (LV1-6) in the anesthetized rats. The rats are anesthetized by injection (i.p.) of 1 ml/kg of a mixture of ketamine/rompun (ratio of 4 to 3), and then placed on a rat platform. The scan field sized is 6×1.9 cm, resolution is 0.0254×0.0127 cm, and scan speed is 7.25 mm/sec. The whole lumbar spine scan image is obtained and analyzed. Bone area (BA), and bone mineral content (BMC) is determined, and bone mineral density is calculated (MBC divided by BA) for the whole lumbar spine and each of the six lumbar vertebrae (LV1-6).

Proximal tibial metaphyseal cancellous bone histomorphometric analyses are performed as described above for in the estrogen agonist/antagonist protocol.

Measurements and calculations related to trabecular bone volume and structure are performed as described above in the estrogen agonist/antagonist protocol. Further, measurements and calculations related to bone resorption are also performed as described above in the estrogen agonist/antagonist protocol. Still further, measurements and calculations related to bone formation and turnover are performed as described above in the estrogen agonist/antagonist protocol. Further still, the data obtained is analyzed using the statistical manipulations described above in the estrogen agonist/antagonist protocol.

### Kidney Regeneration Assay

The role of an prostaglandin agonist in kidney regeneration is investigated by the ability of Prostaglandin E₂ (PGE₂) or a prostaglandin agonist to induce the expression of Bone Morphogenetic Protein 7 (BMP-7) in wild type 293S cells and in 293S cells transfected with EP₂.

Methods: 293S and EP2 293S cells are grown in Dulbecco's Modified Eagle medium (DMEM, Gibco, BRL; Gaithersburg, Md.). One day prior to treatment with PGE₂ or an prostaglandin agonist, cells are plated at a density of 1.5×10⁶ cells/10 cm dish. Generally about 16 to 24 hours later the cell monolayer is washed once with OptiMEM (Gibco, BRL; Gaithersburg, Md.) followed by the addition of 10 ml OptiMEM/dish in the presence and absense of vehicle (DMSO), PGE₂ (10⁻⁶M) or a prostaglandin agonist (10⁻⁶M). Cells are harvested and RNA is extracted at 8, 16 and 24 hours. Northern blot analysis of total RNA (20 mg/lane) is carried out by probing the blots with ³²P-labeled BMP-7 probe. The blots are normalized for RNA loading by hybridization with ³²P-labeled 18s ribosomal RNA probe. PGE₂ and prostaglandin agonists induce the expression of BMP-7 in the EP₂ 293S cells in a time dependent manner. Such induction of expression is generally not observed in the parental cell line. Given the known role of BMP-7 in kidney regeneration and the ability of an prostaglandin agonist to induce BMP-7 expression in 293S kidney cells in a time and receptor specific manner indicates a role for prostaglandin agonist in kidney regeneration.

Administration of the compounds of this invention can be via any method which delivers a compound of this invention systemically and/or locally (e.g., at the site of the bone fracture, osteotomy, or orthopedic surgery). These methods include oral routes, parenteral, intraduodenal routes, etc. Generally, the compounds of this invention are administered orally, but parenteral administration (e.g., intravenous, intramuscular, transdermal, subcutaneous, rectal or intramedullary) may be utilized, for example, where oral administration is inappropriate for the target or where the patient is unable to ingest the drug.

The compounds are used for the treatment and promotion of healing of bone fractures and osteotomies by the local application (e.g., to the sites of bone fractures of osteotomies) of the compounds of this invention or compositions thereof. The compounds of this invention are applied to the sites of bone fractures or osteotomies, for example, either by injection of the compound in a suitable solvent (e.g., an oily solvent such as arachis oil) to the cartilage growth plate or, in cases of open surgery, by local application thereto of such compounds in a suitable carrier or diluent such as bone-wax, demineralized bone powder, polymeric bone cements, bone sealants, etc. Alternatively, local application can be achieved by applying a solution or dispersion of the compound in a suitable carrier or diluent onto the surface of, or incorporating it into solid or semi-solid implants conventionally used in orthopedic surgery, such as dacron-mesh, gel-foam and kiel bone, or prostheses.

The compounds of this invention may also be applied locally to the site of the fracture or osteotomy in a suitable carrier or diluent in combination with one or more of the anabolic agents or bone anti-resorptive agents described above.

Such combinations within the scope of this invention can be co-administered simultaneously or sequentially in any order, or a single pharmaceutical composition comprising a polymorphic form of (3-(((4-tert-butyl-benzyl)-(pyridine-3-sulfonyl)-amino)-methyl)-phenoxy)-acetic acid and its sodium salt, a prodrug thereof or a pharmaceutical salt of said compound or said prodrug as described above and a second compound as described above in a pharmaceutically acceptable carrier or diluent can be administered.

For example, a bone anabolic agent can be used in this invention alone or in combination with an anti-resorptive agent for three months to three years, followed by an anti-resorptive agent alone for three months to three years, with optional repeat of the full treatment cycle. Alternatively, for example, the bone anabolic agent can be used alone or in combination with an anti-resorptive agent for three months to three years, followed by an anti-resorptive agent alone for the remainder of the patient's life. For example, in one preferred mode of administration, a polymorphic form of (3-(((4-tert-butyl-benzyl)-(pyridine-3-sulfonyl)-amino)-methyl)-phenoxy)-acetic acid and its sodium salt, or a prodrug thereof or a pharmaceutically acceptable salt of the prodrug as described above may be administered once daily and a second compound as described above (e.g., estrogen agonist/antagonist) may be administered daily in single or multiple doses. Alternatively, for example, in another preferred mode of administration the two compounds may be administered sequentially wherein the polymorphic form of (3-(((4-tert-butyl-benzyl)-(pyridine-3-sulfonyl)-amino)-methyl)-phenoxy)-acetic acid and its sodium salt compound, prodrug thereof or pharmaceutically acceptable salt of the prodrug as described above may be administered once daily for a period of time sufficient to augment bone mass to a level which is above the bone fracture threshold (World Health Organization Study “Assessment of Fracture Risk and its Application to Screening for Postmenopausal Osteoporosis (1994). Report of a World Health Organization Study Group. World Health Organization Technical Series 843”) followed by administration of a second compound, as described above (e.g., estrogen agonist/antagonist), daily in single or multiple doses. It is preferred that the first compound as described above is administered once daily in a rapid delivery form such as oral delivery.

In any event, the amount and timing of compounds administered will, of course, be dependent on the subject being treated, on the severity of the affliction, on the manner of administration and on the judgment of the prescribing physician. Thus, because of patient to patient variability, the dosages given below are a guideline and the physician may titrate doses of the drug to achieve the treatment (e.g., bone mass augmentation) that the physician considers appropriate for the patient. In considering the degree of treatment desired, the physician must balance a variety of factors such as bone mass starting level, age of the patient, presence of preexisting disease, as well as presence of other diseases (e.g., cardiovascular disease).

In general an amount of a compound of this invention is used that is sufficient to augment bone mass to a level which is above the bone fracture threshold (as detailed in the World Health Organization Study previously cited herein).

In general an effective dosage for the anabolic agents used in this invention described above is in the range of 0.001 to 100 mg/kg/day, preferably 0.01 to 50 mg/kg/day.

The following paragraphs provide preferred dosage ranges for various anti-resorptive agents.

The amount of the anti-resorptive agent to be used is determined by its activity as a bone loss inhibiting agent. This activity is determined by means of the pharmacokinetics of an individual compound and its minimal versus maximal effective dose in inhibition of bone loss using a protocol such as described above (e.g., Estrogen Agonist/Antagonist Protocol).

In general, an effective dosage for an anti-resorptive agent is about 0.001 mg/kg/day to about 20 mg/kg/day.

In general, an effective dosage for progestins is about 0.1 to 10 mg per day; the preferred dose is about 0.25 to 5 mg per day.

In general, an effective dosage for polyphosphonates is determined by its potency as a bone resorption inhibiting agent of standard assays.

Ranges for the daily administration of some polyphosphonates are about 0.001 mg/kg/day to about 20 mg/kg/day.

In general an effective dosage for the treatment of this invention, for example the bone resorption treatment of this invention, for the estrogen agonists/antagonists of this invention is in the range of 0.01 to 200 mg/kg/day, preferably 0.5 to 100 mg/kg/day.

In particular, an effective dosage for droloxifene is in the range of 0.1 to 40 mg/kg/day, preferably 0.1 to 5 mg/kg/day.

In particular, an effective dosage for raloxifene is in the range of 0.1 to 100 mg/kg/day, preferably 0.1 to 10 mg/kg/day.

In particular, an effective dosage for tamoxifen is in the range of 0.1 to 100 mg/kg/day, preferably 0.1 to 5 mg/kg/day.

In particular, an effective dosage for 2-(4-methoxy-phenyl)-3-[4-(2-piperidin-1-yl-ethoxy)-phenoxy]-benzo[b]thiophen-6-ol is 0.001 to 1 mg/kg/day.

In particular, an effective dosage for

cis-6-(4-fluoro-phenyl)-5-(4-(2-piperidin-1-yl-ethoxy)-phenyl)-5,6,7,8-tetrahydro-naphthalene-2-ol;

(−)-cis-6-phenyl-5-(4-(2-pyrrolidin-1-yl-ethoxy)-phenyl)-5,6,7,8-tetrahydro-naphthalene-2-ol;

cis-6-phenyl-5-(4-(2-pyrrolidin-1-yl-ethoxy)-phenyl)-5,6,7,8-tetrahydro-naphthalene-2-ol;

cis-1-(6′-pyrrolodinoethoxy-3′-pyridyl)-2-phenyl-6-hydroxy-1,2,3,4-tetrahydronaphthalene;

1-(4′-pyrrolidinoethoxyphenyl)-2-(4″-fluorophenyl)-6-hydroxy-1,2,3,4-tetrahydroisoquinoline;

cis-6-(4-hydroxyphenyl)-5-(4-(2-piperidin-1-yl-ethoxy)-phenyl)-5,6,7,8-tetrahydro-naphthalene-2-ol; or

1-(4′-pyrrolidinolethoxyphenyl)-2-phenyl-6-hydroxy-1,2,3,4-tetrahydroisoquinoline

is in the range of 0.0001 to 100 mg/kg/day, preferably 0.001 to 10 mg/kg/day.

In particular, an effective dosage for 4-hydroxy tamoxifen is in the range of 0.0001 to 100 mg/kg/day, preferably 0.001 to 10 mg/kg/day.

The compounds of the present invention are generally administered in the form of a pharmaceutical composition comprising at least one of the compounds of this invention together with a pharmaceutically acceptable vehicle or diluent. Thus, the compounds of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions of the invention may contain 0.1%-95% of the compound(s) of this invention, preferably 1%-70%. In any event, the composition or Formulation to be administered will contain a quantity of a compound(s) of this invention in an amount effective to treat the disease/condition of the subject being treated, e.g., a bone disorder.

Since the present invention has an aspect that relates to the augmentation and maintenance of bone mass by treatment with a combination of active ingredients which may be administered separately, the invention also relates to combining separate pharmaceutical compositions in kit form. The kit comprises two separate pharmaceutical compositions: a compound of Formula I a prodrug thereof or a pharmaceutically acceptable salt of said compound or said prodrug and a second compound as described above. The kit comprises container means for containing the separate compositions such as a divided bottle or a divided foil packet, however, the separate compositions may also be contained within a single, undivided container. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several tablets or capsules to be taken on a given day. Also, a daily dose of a Formula I compound, a prodrug thereof or a pharmaceutically acceptable salt of said compound or said prodrug can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of this invention either alone or in combination with each other or other compounds generally will be administered in a convenient Formulation. The following Formulation examples only are illustrative and are not intended to limit the scope of the present invention.

In the Formulations which follow, “active ingredient” means a compound or compounds of this invention.

### Formulation 1: Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

A tablet Formulation is prepared using the ingredients below:

### Formulation 2: Tablets

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

### Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution | 4 |
| in water) | |
| Sodium carboxymethyl cellulose | 4.5 |
| Magnesium stearate | 0.5 |
| Talc | 1 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

### Formulation 4: Suspensions

| | Quantity | |
|---|---|---|
| Ingredient | (mg/5 ml) | |
| Active ingredient | 0.25-100 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with stirring. Sufficient water is then added to produce the required volume.

An aerosol solution is prepared containing the following ingredients:

### Formulation 5: Aerosol

| | Quantity |
|---|---|
| Ingredient | (% by weight) |
| Active ingredient | 0.25 |
| Ethanol | 25.75 |
| Propellant 22 (Chlorodifluoromethane) | 70.00 |

The active ingredient is mixed with ethanol and the mixture added to a portion of the propellant, cooled to 30° C., and transferred to a filling device. The required amount is then fed to a stainless steel container and diluted with the remaining propellant. The valve units are then fitted to the container.

Suppositories are prepared as follows:

### Formulation 6: Suppositories

| Ingredient | Quantity (mg/suppository) |
|---|---|
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous Formulation is prepared as follows:

### Formulation 7: Intravenous Solution

| Ingredient | Quantity |
|---|---|
| Active ingredient | 20 mg |
| Isotonic saline | 1,000 mL |

The solution of the above ingredients is intravenously administered to a patient at a rate of about 1 mL per minute.

The active ingredient above may also be a combination of agents.

It should be understood, of course, that the foregoing relates to exemplary embodiments of the invention and that modifications may be made without departing from the spirit and scope of the invention as set forth in the following claims.

1. A crystalline form of (3-(((4-tert-butyl-benzyl)-(pyridine-3-sulfonyl)-amino)-methyl)-phenoxy)-acetic acid sodium salt or a hydrate thereof selected from crystalline forms A and C.

2. The crystalline form according to claim 1, wherein:
the crystalline form is crystalline form A; and
the X-ray powder diffraction pattern contains the following 2θ values measured using CuK_{α} radiation: 3.2, 4.1, 20.2 and 20.8.

3. The crystalline form according to claim 1, wherein:
the crystalline form is crystalline form A; and
the X-ray powder diffraction pattern contains the following 2θ values measured using CuK_{α} radiation: 3.2, 4.1, 16.1, 20.2 and 20.8.

4. The crystalline form according claim 1, wherein:
the crystalline form is crystalline form A; and
the crystalline form contains 0.5 moles of water per mole of crystalline form.

5.-7. (canceled)

8. The crystalline form according to claim 1, wherein:
the crystalline form is crystalline form C; and
the X-ray powder diffraction pattern contains the following 2θ values measured using CuK_{α} radiation: 3.6, 4.0 and 13.3.

9. The crystalline form according to claim 1, wherein:
the crystalline form is crystalline form C; and
the X-ray powder diffraction pattern contains the following 2θ values measured using CuK_{α} radiation: 3.5, 4.0, 5.9, 13.3 and 16.9.

10. The crystalline form according to claim 1, wherein:
the crystalline form is crystalline form C; and
the crystalline form contains 1.0 mole of water per mole of crystalline form.

11.-16. (canceled)

17. The crystalline form according to claim 1, wherein:
the crystalline form is crystalline form A; and
the crystalline form has a solid state ¹³C nuclear magnetic resonance having the following chemical shifts expressed in parts per millions: 31.2, 32.1, 33.7 and 34.5.

18. The crystalline form according to claim 17, wherein the crystalline form has a solid state ¹³C nuclear magnetic resonance having the following chemical shifts expressed in parts per millions: 31.2, 32.1, 33.7, 34.5, 173.2, 176.3, and 178.2.

19. The crystalline form according to claim 1, wherein:
the crystalline form is crystalline form A; and
the crystalline form has a solid state ¹³C nuclear magnetic resonance having the following chemical shifts expressed in parts per millions: 173.2, 176.3, and 178.2.

20. A substantially pure crystalline form A or form C of (3-(((4-tert-butyl-benzyl)-(pyridine-3-sulfonyl)-amino)-methyl)-phenoxy)-acetic acid sodium salt or a hydrate thereof.

21. (canceled)

22. Crystalline form A of (3-(((4-tert-butyl-benzyl)-(pyridine-3-sulfonyl)-amino)-methyl)-phenoxy)-acetic acid sodium salt hemi-hydrate.

23.-25. (canceled)

26. A non-crystalline form of (3-(((4-tert-butyl-benzyl)-(pyridine-3-sulfonyl)-amino)-methyl)-phenoxy)-acetic acid sodium salt or a hydrate thereof, wherein
the non-crystalline form is non-crystalline form H; and
the X-ray powder diffraction pattern contains the following 2θ values measured using CuK_{α} radiation: 3.1.

27. The non-crystalline form according to claim 26, wherein:
the non-crystalline form has a solid state ¹³C nuclear magnetic resonance having the following chemical shifts expressed in parts per millions: 125.7, 151.2, and 175.8.

28. An amorphous form of (3-(((4-tert-butyl-benzyl)-(pyridine-3-sulfonyl)-amino)-methyl)-phenoxy)-acetic acid sodium salt or a hydrate thereof, wherein
the amorphous form is amorphous Form I; and
the X-ray powder diffraction pattern is substantially as shown in FIG. 6C.
